SEQUENCE LISTING (10 sequences)

SEQ ID NO: 1 - DNA, 2771 bp, Homo sapiens
  misc_feature (1)..(2771): gi15619005;  NM_004624.2;  VIPR1
ggccacaggccagcgccactctgccaggctcccggccatcgcccgcctggtgcgccgcccgccagctctttgcccgcgcggggccgcccgccgcgggctcagggcagaccatgcgcccgccaagtccgctgcccgcccgctggctatgcgtgctggcaggcgccctcgcctgggcccttgggccggcgggcggccaggcggccaggctgcaggaggagtgtgactatgtgcagatgatcgaggtgcagcacaagcagtgcctggaggaggcccagctggagaatgagacaataggctgcagcaagatgtgggacaacctcacctgctggccagccacccctcggggccaggtagttgtcttggcctgtcccctcatcttcaagctcttctcctccattcaaggccgcaatgtaagccgcagctgcaccgacgaaggctggacgcacctggagcctggcccgtaccccattgcctgtggtttggatgacaaggcagcgagtttggatgagcagcagaccatgttctacggttctgtgaagaccggctacaccattggctacggcctgtccctcgccacccttctggtcgccacagctatcctgagcctgttcaggaagctccactgcacgcggaactacatccacatgcacctcttcatatccttcatcctgagggctgccgctgtcttcatcaaagacttggccctcttcgacagcggggagtcggaccagtgctccgagggctcggtgggctgtaaggcagccatggtctttttccaatattgtgtcatggctaacttcttctggctgctggtggagggcctctacctgtacaccctgcttgccgtctccttcttctctgagcggaagtacttctgggggtacatactcatcggctggggggtacccagcacattcaccatggtgtggaccatcgccaggatccattttgaggattatgggtgctgggacaccatcaactcctcactgtggtggatcataaagggccccatcctcacctccatcttggtaaacttcatcctgtttatttgcatcatccgaatcctgcttcagaaactgcggcccccagatatcaggaagagtgacagcagtccatactcaaggctagccaggtccacactcctgctgatccccctgtttggagtacactacatcatgttcgccttctttccggacaattttaagcctgaagtgaagatggtctttgagctcgtcgtggggtctttccagggttttgtggtggctatcctctactgcttcctcaatggtgaggtgcaggcggagctgaggcggaagtggcggcgctggcacctgcagggcgtcctgggctggaaccccaaataccggcacccgtcgggaggcagcaacggcgccacgtgcagcacgcaggtttccatgctgacccgcgtcagcccaggtgcccgccgctcctccagcttccaagccgaagtctccctggtctgaccaccaggatcccaggggcccaaggcggcccctcccgccccttcccactcaccccggcagacgccggggacagaggcctgcccgggcgcggccagccccggccctgggctcggaggctgcccccggccccctggtctctggtccggacactcctagagaacgcagccctagagcctgcctggagcgtttctagcaagtgagagagatgggagctcctctcctggaggattgcaggtggaactcagtcattagactcctcctccaaaggccccctacgccaatcaagggcaaaaagtctacatactttcatcctgactctgccccctgctggctcttctgcccaattggaggaaagcaaccggtggatcctcaaacaacactggtgtgacctgagggcagaaaggttctgcccggggaaggtcaccagcaccaacaccacggtagtgcctgaaatttcaccattgctgtcaagttcctttgggttaagcattaccactcaggcatttgactgaagatgcagctcactaccctattctctctttacgcttagttatcagctttttaaagtgggttattctggagtttttgtttggagagcacacctatcttagtggttccccaccgaagtggactggcccctgggtcagtctggtgggaggacggtgcaacccaaggactgagggactctgaagcctctgggaaatgagaaggcagccaccagcgaatgctaggtctcggactaagcctacctgctctccaagtctcagtggcttcatctgtcaagtgggatctgtcacaccagccatacttatctctctgtgctgtggaagcaacaggaatcaagagctgccctccttgtccacccacctatgtgccaactgttgtaactaggctcagagatgtgcacccatgggctctgacagaaagcagatacctcaccctgctacacatacaggatttgaactcagatctgtctgataggaatgtgaaagcacggactcttactgctaacttttgtgtatcgtaaccagccagatcctcttggttatttgtttaccacttgtattattaatgccattatcctgaattccccttgccaccccaccctccctggcgtgtggctgaggaggcctccatctcatgtatcatctggataggagcctgctggtcacagcctcctctgtctgcccttcaccccagtggccactcagcttcctacccacacctctgccagaagatcccctcaggactgcaacaggcttgtgcaacaataaatgttggcttgga

SEQ ID NO: 2 - protein, 457 aa, Homo sapiens
  MISC_FEATURE (1)..(457): gi15619006;  NP_004615.2;  VIPR1
MRPPSPLPARWLCVLAGALAWALGPAGGQAARLQEECDYVQMIEVQHKQCLEEAQLENETIGCSKMWDNLTCWPATPRGQVVVLACPLIFKLFSSIQGRNVSRSCTDEGWTHLEPGPYPIACGLDDKAASLDEQQTMFYGSVKTGYTIGYGLSLATLLVATAILSLFRKLHCTRNYIHMHLFISFILRAAAVFIKDLALFDSGESDQCSEGSVGCKAAMVFFQYCVMANFFWLLVEGLYLYTLLAVSFFSERKYFWGYILIGWGVPSTFTMVWTIARIHFEDYGCWDTINSSLWWIIKGPILTSILVNFILFICIIRILLQKLRPPDIRKSDSSPYSRLARSTLLLIPLFGVHYIMFAFFPDNFKPEVKMVFELVVGSFQGFVVAILYCFLNGEVQAELRRKWRRWHLQGVLGWNPKYRHPSGGSNGATCSTQVSMLTRVSPGARRSSSFQAEVSLV

SEQ ID NO: 3 - RNA, 21 nt, Homo sapiens
  misc_feature (1)..(21): VIPR1-PO5 oligonucleotide
gcacgcagguuuccaugcuuu

SEQ ID NO: 4 - RNA, 21 nt, Homo sapiens
  misc_feature (1)..(21): VIPR1-PO6 oligonucleotide
gacaauaggcugcagcaaguu

SEQ ID NO: 5 - RNA, 21 nt, Homo sapiens
  misc_feature (1)..(21): VIPR1-P07 oligonucleotide
ucacugugguggaucauaauu

SEQ ID NO: 6 - RNA, 21 nt, Homo sapiens
  misc_feature (1)..(21): VIPR1-P08 oligonucleotide
aauauugugucauggcuaauu

SEQ ID NO: 7 - RNA, 19 nt, Homo sapiens
  misc_feature (1)..(19): VIPR1-SO1 oligonucleotide
ggagagcacaccuaucuua

SEQ ID NO: 8 - RNA, 19 nt, Homo sapiens
  misc_feature (1)..(19): VIPR1-S02 oligonucleotide
gcaacaggcuugugcaaca

SEQ ID NO: 9 - RNA, 19 nt, Homo sapiens
  misc_feature (1)..(19): VIPR1-S03 oligonucleotide
acaauaggcugcagcaaga

SEQ ID NO: 10 - RNA, 19 nt, Homo sapiens
  misc_feature (1)..(19): VIPR1-S04 oligonucleotide
ucacugugguggaucauaa

(19) United States
(12) Patent Application Publication
(10) Pub. No.: US 2010/0112566 A1
(43) Pub. Date: May 6, 2010

(54) VIPR1S as Modifiers of the E2F/RB Pathway and Methods of Use

(76) Inventors: Kyle A. Edgar, Berkeley, CA (US); Kimberly Carr Ferguson, El Granada, CA (US); Monique Nicoll, El Granada, CA (US); Christopher G. Winter, Needham, MA (US)

Correspondence Address: MCDONNELL BOEHNEN HULBERT @ BERGHOFF LLP, 300 SOUTH WACKER DRIVE, SUITE 3100, CHICAGO, IL, 60606, US

(21) Appl. No.: 12/442,372

(22) PCT Filed: Sep. 24, 2007
(86) PCT No.: PCT/US07/20667

§ 371 Date: Nov. 6, 2009

Related U.S. Application Data
(60) Provisional application No. 60/846,860, filed on Sep. 22, 2006.

(51) Int. Cl. C12Q 1/68; G01N 33/53

(52) U.S. Cl. 435/6; 435/7.2

(57) ABSTRACT

Human VIPR1 genes are identified as modulators of the E2F/RB pathway, and thus are therapeutic targets for disorders associated with defective E2F/RB function. Methods for identifying modulators of E2F/RB, comprising screening for agents that modulate the activity of VIPR1 are provided.

This application is the U.S. National Phase of PCT/US2007/020667 which claims priority to application Ser. No. 60/846,860, filed Sep. 22, 2006, which is incorporated herein by reference in its entirety.

### BACKGROUND OF THE INVENTION

The E2F signaling pathway is frequently hyperactivated by a variety of mechanisms in a wide range of human cancers, including lung, breast, glioblastoma, pancreatic and soft tissue sarcomas (see Malumbres M and Barbacid M (2001) Nat Rev Cancer. 1: 222-231; Ortega S et al. (2002) Biochim et Biophyis Acta. 1602: 73-87). In tumor 10 cells, E2F activity can be elevated by deletion of the CDK4/6 inhibitor p16INK4, amplification of the CDK4/6 or D-type cyclin loci, or mutation of the negative regulator pRb, which allows constitutive activation of E2F transcription of critical G1/S phase genes and progression through the cell cycle. Normal cells have intact checkpoints that are regulated at critical phases of the cell cycle such that DNA replication and cell division occur only in the presence of mitogenic stimuli and appropriate nutrient availability. One of the critical regulatory decision points is between the G1/S transition, and in non cycling cells, the activity of the CDK4/6/cyclin D complex is inhibited, the pRb tumor suppressor is hypophosphorylated allowing pRb to bind to and inhibit E2F transcription factors from transcribing gene products that are required for DNA replication. E2F transcription can be de-regulated in tumors by deletion or mutation of the p16INK4 gene, which normally would bind to and inhibit CDK4/6/cyclinD complexes from forming and phosphorylating and inactivating pRb. This loss occurs frequently, e.g. in 60% of glioblastomas and 80% of pancreatic cancers. In addition, in some tumors, CDK4/6 kinase activity is increased by gene amplification of the CDKs themselves, or of the D-type cyclin genes. In addition, tumors often harbor mutations in pRb that result in loss of function of this critical tumor suppressor (e.g. in up to 80% of small cell lung cancer tumors). The E2F pathway is likely to promote tumor progression by its well described role in promoting cell proliferation, but in addition there have been recent reports that E2F regulates genes involved promoting progress through G2/M, as well as genes regulating apoptosis and DNA damage repair which also could contribute to E2F driven tumor progression (see Bracken A et al, (2004) Trends in Biochemical Sciences. 29(8): 409-417.

All references cited herein, including patents, patent applications, publications, and sequence information in referenced Genbank identifier numbers, are incorporated herein in their entireties.

### SUMMARY OF THE INVENTION

We have discovered genes that modify the E2F/RB pathway hereinafter referred to as Modifiers of E2F/RB (ME2F/RB). Specifically, we have discovered that one gene, Vasoactive Intestinal Peptide Receptor 1 (VIPR1) modifies the E2F/RB pathway in a number of human tissues and cell lines. The invention provides methods for utilizing these E2F/RB modifier genes and polypeptides to identify VIPR1-modulating agents that are candidate therapeutic agents that can be used in the treatment of disorders associated with defective or impaired E2F/RB function and/or VIPR1 function. Preferred VIPR1-modulating agents specifically bind to VIPR1 polypeptides and restore E2F/RB function. Other preferred VIPR1-modulating agents are nucleic acid modulators such as antisense oligomers and RNAi that repress VIPR1 gene expression or product activity by, for example, binding to and inhibiting the respective nucleic acid (i.e. DNA or mRNA).

VIPR1 modulating agents may be evaluated by any convenient in vitro or in vivo assay for molecular interaction with a VIPR1 polypeptide or nucleic acid. In one embodiment, candidate VIPR1 modulating agents are tested with an assay system comprising an VIPR1 polypeptide or nucleic acid. Agents that produce a change in the activity of the assay system relative to controls are identified as candidate E2F/RB modulating agents. The assay system may be cell-based or cell-free. VIPR1-modulating agents include VIPR1 related proteins (e.g. dominant negative mutants, and biotherapeutics); VIPR1-specific antibodies; VIPR1-specific antisense oligomers and other nucleic acid modulators; and chemical agents that specifically bind to or interact with VIPR1 or compete with VIPR1 binding partner (e.g. by binding to an VIPR1 binding partner). In one specific embodiment, a small molecule modulator is identified using a binding assay. In specific embodiments, the screening assay system is selected from an apoptosis assay, a cell proliferation assay, and an angiogenesis assay.

In another embodiment, candidate E2F/RB pathway modulating agents are further tested using a second assay system that detects changes in the E2F/RB pathway, such as protein phosphorylation, cell cycle progression or cell proliferation changes produced by the originally identified candidate agent or an agent derived from the original agent. The second assay system may use cultured cells or non-human animals. In specific embodiments, the secondary assay system uses non-human animals, including animals predetermined to have a disease or disorder implicating the E2F/RB pathway, such as an angiogenic, apoptotic, cell cycle, or cell proliferation disorder (e.g. cancer).

The invention further provides methods for modulating the VIPR1 function and/or the E2F/RB pathway in a mammalian cell by contacting the mammalian cell with an agent that specifically binds a VIPR1 polypeptide or nucleic acid. The agent may be a small molecule modulator, a nucleic acid modulator, or an antibody and may be administered to a mammalian animal predetermined to have a pathology associated with the E2F/RB pathway.

### DETAILED DESCRIPTION OF THE INVENTION

A genetic screen was designed which employed RNAi of specific genes to identify genetic modifiers of E2F/RB pathway function in the non-small-lung cell cancer cell line NCI-H1299. Methods for using RNAi to silence genes in are known in the art (Fire A, et al., 1998 Nature 391:806-811; Fire, A. Trends Genet. 15, 358-363 (1999); WO9932619).

Genes causing altered phenotypes in the NCI-H1299 cells were identified as modifiers of the E2F/RB pathway. Specifically, we have discovered that one gene, Vasoactive Intestinal Peptide Receptor 1 (VIPR1) modifies the E2F/RB pathway in a number of human tissues and cell lines. The VIPR1 gene is located on 3p22 in the human genome and it encodes a 457 aa transmembrane protein. The protein contains an extracellular hormone receptor domain that contains 4 concerved cysteine residues that probably form disulfide bridges, 7 transmembrane domains and an intracellular domain. The VIPR1 protein is a class II GPCR (G protein coupled receptor) that binds to a number of ligands including but not limited to VIP (vasoactive intestinal peptide), glucagon, secretin, and GHRP (growth hormone releasing peptide). VIP has many functions in the circulatory, immune, reproductive and GI systems such as hormone release, muscle relaxation, metabolism, immune functions, fetal growth. VIPR1 is found predominantly in lung, small intestine, thymus, and brain. The VIPR1 gene is expressed on many tumor cells and is identified using labeled ligands in tumor imaging and diagnostic procedures. The VIPR1 gene is expressed on such tumor cell lines as the H1299, CALU-6, H727, A549 and H838 NSCLC (non-small cell lung carcinomas), the SK-Br-3, MCF-7, MDA-MB231T, and MDAMB468 breast tumor cell lines, the HT-29 and SW480 colon cancer cell lines and the PANC1 pancreatic cancer cell lines. Inhibition or modulation of VIPR1 expression or protein activity can inhibit or modulate the cAMP response in tissues and cells. VIPR1 siRNA are known to inhibit VIP binding to cells affecting the cAMP response, cell proliferation especially in H1299 cells. siRNAs directed against VIPR1 were also shown to affect cell proliferation in Calu6 and H838 tumor cell lines. siRNAs directed against VIPR1 were shown to have an effect in phenotypic and pathway assays such as decreasing Brdu incorporation, decreasing cell counts, increasing Caspase 3, and altering the Rb phosphorylation ratio in H1299 cells. Accordingly modifiers of VIPR1 genes (i.e., nucleic acids and polypeptides) are attractive drug targets for the treatment of pathologies associated with a defective E2F/RB signaling pathway, such as cancer. Table 1 (Example II) lists VIPR1 orthologs and aliases.

In vitro and in vivo methods of assessing VIPR1 function are provided herein. Modulation of the VIPR1 or their respective binding partners is useful for understanding the association of the E2F/RB pathway and its members in normal and disease conditions and for developing diagnostics and therapeutic modalities for E2F/RB related pathologies. VIPR1-modulating agents that act by inhibiting or enhancing VIPR1 expression, directly or indirectly, for example, by affecting an VIPR1 function such as enzymatic (e.g., catalytic) or binding activity, can be identified using methods provided herein. VIPR1 modulating agents are useful in diagnosis, therapy and pharmaceutical development.

Nucleic Acids and Polypeptides of the Invention

Sequences related to VIPR1 nucleic acids and polypeptides that can be used in the invention are disclosed in Genbank (referenced by Genbank identifier (GI) or RefSeq number), shown in Table 1 and in the appended sequence listing.

The term “VIPR1 polypeptide” refers to a full-length VIPR1 protein or a functionally active fragment or derivative thereof A “functionally active” VIPR1 fragment or derivative exhibits one or more functional activities associated with a full-length, wild-type VIPR1 protein, such as antigenic or immunogenic activity, enzymatic activity, ability to bind natural cellular substrates, etc. The functional activity of VIPR1 proteins, derivatives and fragments can be assayed by various methods known to one skilled in the art (Current Protocols in Protein Science (1998) Coligan et al., eds., John Wiley & Sons, Inc., Somerset, N.J.) and as further discussed below. In one embodiment, a functionally active VIPR1 polypeptide is a VIPR1 derivative capable of rescuing defective endogenous VIPR1 activity, such as in cell based or animal assays; the rescuing derivative may be from the same or a different species. For purposes herein, functionally active fragments also include those fragments that comprise one or more structural domains of a VIPR1, such as a binding domain. Protein domains can be identified using the PFAM program (Bateman A., et al., Nucleic Acids Res, 1999, 27:260-2). Methods for obtaining VIPR1 polypeptides are also further described below. In some embodiments, preferred fragments are functionally active, domain-containing fragments comprising at least 25 contiguous amino acids, preferably at least 50, more preferably 75, and most preferably at least 100 contiguous amino acids of a VIPR1. In further preferred embodiments, the fragment comprises the entire functionally active domain.

The term “VIPR1 nucleic acid” refers to a DNA or RNA molecule that encodes a VIPR1 polypeptide. Preferably, the VIPR1 polypeptide or nucleic acid or fragment thereof is from a human, but can also be an ortholog, or derivative thereof with at least 70% sequence identity, preferably at least 80%, more preferably 85%, still more preferably 90%, and most preferably at least 95% sequence identity with human VIPR1. Methods of identifying orthlogs are known in the art. Normally, orthologs in different species retain the same function, due to presence of one or more protein motifs and/or 3-dimensional structures. Orthologs are generally identified by sequence homology analysis, such as BLAST analysis, usually using protein bait sequences. Sequences are assigned as a potential ortholog if the best hit sequence from the forward BLAST result retrieves the original query sequence in the reverse BLAST (Huynen M A and Bork P, Proc Natl Acad Sci (1998) 95:5849-5856; Huynen M A et al., Genome Research (2000) 10:1204-1210). Programs for multiple sequence alignment, such as CLUSTAL (Thompson J D et al, 1994, Nucleic Acids Res 22:4673-4680) may be used to highlight conserved regions and/or residues of orthologous proteins and to generate phylogenetic trees. In a phylogenetic tree representing multiple homologous sequences from diverse species (e.g., retrieved through BLAST analysis), orthologous sequences from two species generally appear closest on the tree with respect to all other sequences from these two species. Structural threading or other analysis of protein folding (e.g., using software by ProCeryon, Biosciences, Salzburg, Austria) may also identify potential orthologs. In evolution, when a gene duplication event follows speciation, a single gene in one species, such as C. elegans, may correspond to multiple genes (paralogs) in another, such as human. As used herein, the term “orthologs” encompasses paralogs. As used herein, “percent (%) sequence identity” with respect to a subject sequence, or a specified portion of a subject sequence, is defined as the percentage of nucleotides or amino acids in the candidate derivative sequence identical with the nucleotides or amino acids in the subject sequence (or specified portion thereof), after aligning the sequences and introducing gaps, if necessary to achieve the maximum percent sequence identity, as generated by the program WU-BLAST-2.0a19 (Altschul et al., J. Mol. Biol. (1997) 215:403-410) with all the search parameters set to default values. The HSP S and HSP S2 parameters are dynamic values and are established by the program itself depending upon the composition of the particular sequence and composition of the particular database against which the sequence of interest is being searched. A % identity value is determined by the number of matching identical nucleotides or amino acids divided by the sequence length for which the percent identity is being reported. “Percent (%) amino acid sequence similarity” is determined by doing the same calculation as for determining % amino acid sequence identity, but including conservative amino acid substitutions in addition to identical amino acids in the computation.

A conservative amino acid substitution is one in which an amino acid is substituted for another amino acid having similar properties such that the folding or activity of the protein is not significantly affected. Aromatic amino acids that can be substituted for each other are phenylalanine, tryptophan, and tyrosine; interchangeable hydrophobic amino acids are leucine, isoleucine, methionine, and valine; interchangeable polar amino acids are glutamine and asparagine; interchangeable basic amino acids are arginine, lysine and histidine; interchangeable acidic amino acids are aspartic acid and glutamic acid; and interchangeable small amino acids are alanine, serine, threonine, cysteine and glycine.

Alternatively, an alignment for nucleic acid sequences is provided by the local homology algorithm of Smith and Waterman (Smith and Waterman, 1981, Advances in Applied Mathematics 2:482-489; database: European Bioinformatics Institute; Smith and Waterman, 1981, J. of Molec. Biol., 147:195-197; Nicholas et al., 1998, “A Tutorial on Searching Sequence Databases and Sequence Scoring Methods” (www.psc.edu) and references cited therein; W. R. Pearson, 1991, Genomics 11:635-650). This algorithm can be applied to amino acid sequences by using the scoring matrix developed by Dayhoff (Dayhoff: Atlas of Protein Sequences and Structure, M. O. Dayhoff ed., 5 suppl. 3:353-358, National Biomedical Research Foundation, Washington, D.C., USA), and normalized by Gribskov (Gribskov 1986 Nucl. Acids Res. 14(6):6745-6763). The Smith-Waterman algorithm may be employed where default parameters are used for scoring (for example, gap open penalty of 12, gap extension penalty of two). From the data generated, the “Match” value reflects “sequence identity.”

Derivative nucleic acid molecules of the subject nucleic acid molecules include sequences that hybridize to the nucleic acid sequence of a VIPR1. The stringency of hybridization can be controlled by temperature, ionic strength, pH, and the presence of denaturing agents such as formamide during hybridization and washing. Conditions routinely used are set out in readily available procedure texts (e.g., Current Protocol in Molecular Biology, Vol. 1, Chap. 2.10, John Wiley & Sons, Publishers (1994); Sambrook et al., Molecular Cloning, Cold Spring Harbor (1989)). In some embodiments, a nucleic acid molecule of the invention is capable of hybridizing to a nucleic acid molecule containing the nucleotide sequence of a VIPR1 under high stringency hybridization conditions that are: prehybridization of filters containing nucleic acid for 8 hours to overnight at 65° C. in a solution comprising 6× single strength citrate (SSC) (1×SSC is 0.15 M NaCl, 0.015 M Na citrate; pH 7.0), 5×Denhardt's solution, 0.05% sodium pyrophosphate and 100 μg/ml herring sperm DNA; hybridization for 18-20 hours at 65° C. in a solution containing 6×SSC, 1×Denhardt's solution, 100 μg/ml yeast tRNA and 0.05% sodium pyrophosphate; and washing of filters at 65° C. for 1 h in a solution containing 0.1×SSC and 0.1% SDS (sodium dodecyl sulfate).

In other embodiments, moderately stringent hybridization conditions are used that are: pretreatment of filters containing nucleic acid for 6 h at 40° C. in a solution containing 35% formamide, 5×SSC, 50 mM Tris-HCl (pH7.5), 5 mM EDTA, 0.1% PVP, 0.1% Ficoll, 1% BSA, and 500 μg/ml denatured salmon sperm DNA; hybridization for 18-20 h at 40° C. in a solution containing 35% formamide, 5×SSC, 50 mM Tris-HCl (pH7.5), 5 mM EDTA, 0.02% PVP, 0.02% Ficoll, 0.2% BSA, 100 μg/ml salmon sperm DNA, and 10% (wt/vol) dextran sulfate; followed by washing twice for 1 hour at 55° C. in a solution containing 2×SSC and 0.1% SDS.

Alternatively, low stringency conditions can be used that are: incubation for 8 hours to overnight at 37° C. in a solution comprising 20% formamide, 5×SSC, 50 mM sodium phosphate (pH 7.6), 5×Denhardt's solution, 10% dextran sulfate, and 20 μg/ml denatured sheared salmon sperm DNA; hybridization in the same buffer for 18 to 20 hours; and washing of filters in 1×SSC at about 37° C. for 1 hour.

Isolation, Production, Expression, and Mis-Expression of VIPR1 Nucleic Acids and Polypeptides

VIPR1 nucleic acids and polypeptides are useful for identifying and testing agents that modulate VIPR1 function and for other applications related to the involvement of VIPR1 in the E2F/RB pathway. VIPR1 nucleic acids and derivatives and orthologs thereof may be obtained using any available method. For instance, techniques for isolating cDNA or genomic DNA sequences of interest by screening DNA libraries or by using polymerase chain reaction (PCR) are well known in the art. In general, the particular use for the protein will dictate the particulars of expression, production, and purification methods. For instance, production of proteins for use in screening for modulating agents may require methods that preserve specific biological activities of these proteins, whereas production of proteins for antibody generation may require structural integrity of particular epitopes. Expression of proteins to be purified for screening or antibody production may require the addition of specific tags (e.g., generation of fusion proteins). Overexpression of a VIPR1 protein for assays used to assess VIPR1 function, such as involvement in cell cycle regulation or hypoxic response, may require expression in eukaryotic cell lines capable of these cellular activities. Techniques for the expression, production, and purification of proteins are well known in the art; any suitable means therefore may be used (e.g., Higgins SJ and Hames BD (eds.) Protein Expression: A Practical Approach, Oxford University Press Inc., New York 1999; Stanbury PF et al., Principles of Fermentation Technology, 2^{nd} edition, Elsevier Science, New York, 1995; Doonan S (ed.) Protein Purification Protocols, Humana Press, New Jersey, 1996; Coligan J E et al, Current Protocols in Protein Science (eds.), 1999, John Wiley & Sons, New York). In particular embodiments, recombinant VIPR1 is expressed in a cell line known to have defective E2F/RB function. The recombinant cells are used in cell-based screening assay systems of the invention, as described further below.

The nucleotide sequence encoding a VIPR1 polypeptide can be inserted into any appropriate expression vector. The necessary transcriptional and translational signals, including promoter/enhancer element, can derive from the native VIPR1 gene and/or its flanking regions or can be heterologous. A variety of host-vector expression systems may be utilized, such as mammalian cell systems infected with virus (e.g. vaccinia virus, adenovirus, etc.); insect cell systems infected with virus (e.g. baculovirus); microorganisms such as yeast containing yeast vectors, or bacteria transformed with bacteriophage, plasmid, or cosmid DNA. An isolated host cell strain that modulates the expression of, modifies, and/or specifically processes the gene product may be used.

To detect expression of the VIPR1 gene product, the expression vector can comprise a promoter operably linked to a VIPR1 gene nucleic acid, one or more origins of replication, and, one or more selectable markers (e.g. thymidine kinase activity, resistance to antibiotics, etc.). Alternatively, recombinant expression vectors can be identified by assaying for the expression of the VIPR1 gene product based on the physical or functional properties of the VIPR1 protein in in vitro assay systems (e.g. immunoassays).

The VIPR1 protein, fragment, or derivative may be optionally expressed as a fusion, or chimeric protein product (i.e. it is joined via a peptide bond to a heterologous protein sequence of a different protein), for example to facilitate purification or detection. A chimeric product can be made by ligating the appropriate nucleic acid sequences encoding the desired amino acid sequences to each other using standard methods and expressing the chimeric product. A chimeric product may also be made by protein synthetic techniques, e.g. by use of a peptide synthesizer (Hunkapiller et al., Nature (1984) 310:105-111).

Once a recombinant cell that expresses the VIPR1 gene sequence is identified, the gene product can be isolated and purified using standard methods (e.g. ion exchange, affinity, and gel exclusion chromatography; centrifugation; differential solubility; electrophoresis). Alternatively, native VIPR1 proteins can be purified from natural sources, by standard methods (e.g. immunoaffinity purification). Once a protein is obtained, it may be quantified and its activity measured by appropriate methods, such as immunoassay, bioassay, or other measurements of physical properties, such as crystallography.

The methods of this invention may also use cells that have been engineered for altered expression (mis-expression) of VIPR1 or other genes associated with the E2F/RB pathway. As used herein, mis-expression encompasses ectopic expression, over-expression, under-expression, and non-expression (e.g. by gene knock-out or blocking expression that would otherwise normally occur).

Genetically Modified Animals

Animal models that have been genetically modified to alter VIPR1 expression may be used in in vivo assays to test for activity of a candidate E2F/RB modulating agent, or to further assess the role of VIPR1 in an E2F/RB pathway process such as apoptosis or cell proliferation. Preferably, the altered VIPR1 expression results in a detectable phenotype, such as decreased or increased levels of cell proliferation, angiogenesis, or apoptosis compared to control animals having normal VIPR1 expression. The genetically modified animal may additionally have altered E2F/RB expression (e.g. E2F/RB knockout). Preferred genetically modified animals are mammals such as primates, rodents (preferably mice or rats), among others. Preferred non-mammalian species include zebrafish, C. elegans, and Drosophila. Preferred genetically modified animals are transgenic animals having a heterologous nucleic acid sequence present as an extrachromosomal element in a portion of its cells, i.e. mosaic animals (see, for example, techniques described by Jakobovits, 1994, Curr. Biol. 4:761-763.) or stably integrated into its germ line DNA (i.e., in the genomic sequence of most or all of its cells). Heterologous nucleic acid is introduced into the germ line of such transgenic animals by genetic manipulation of, for example, embryos or embryonic stem cells of the host animal.

Methods of making transgenic animals are well-known in the art (for transgenic mice see Brinster et al., Proc. Nat. Acad. Sci. USA 82: 4438-4442 (1985), U.S. Pat. Nos. 4,736,866 and 4,870,009, both by Leder et al., U.S. Pat. No. 4,873,191 by Wagner et al., and Hogan, B., Manipulating the Mouse Embryo, Cold Spring Harbor Laboratory Press, Cold Spring Harbor, N.Y., (1986); for particle bombardment see U.S. Pat. No. 4,945,050, by Sandford et al.; for transgenic Drosophila see Rubin and Spradling, Science (1982) 218:348-53 and U.S. Pat. No. 4,670,388; for transgenic insects see Berghammer A. J. et al., A Universal Marker for Transgenic Insects (1999) Nature 402:370-371; for transgenic Zebrafish see Lin S., Transgenic Zebrafish, Methods Mol Biol. (2000); 136:375-3830); for microinjection procedures for fish, amphibian eggs and birds see Houdebine and Chourrout, Experientia (1991) 47:897-905; for transgenic rats see Hammer et al., Cell (1990) 63:1099-1112; and for culturing of embryonic stem (ES) cells and the subsequent production of transgenic animals by the introduction of DNA into ES cells using methods such as electroporation, calcium phosphate/DNA precipitation and direct injection see, e.g., Teratocarcinomas and Embryonic Stem Cells, A Practical Approach, E. J. Robertson, ed., IRL Press (1987)). Clones of the nonhuman transgenic animals can be produced according to available methods (see Wilmut, I. et al. (1997) Nature 385:810-813; and PCT International Publication Nos. WO 97/07668 and WO 97/07669).

In one embodiment, the transgenic animal is a “knock-out” animal having a heterozygous or homozygous alteration in the sequence of an endogenous VIPR1 gene that results in a decrease of VIPR1 function, preferably such that VIPR1 expression is undetectable or insignificant. Knock-out animals are typically generated by homologous recombination with a vector comprising a transgene having at least a portion of the gene to be knocked out. Typically a deletion, addition or substitution has been introduced into the transgene to functionally disrupt it. The transgene can be a human gene (e.g., from a human genomic clone) but more preferably is an ortholog of the human gene derived from the transgenic host species. For example, a mouse VIPR1 gene is used to construct a homologous recombination vector suitable for altering an endogenous VIPR1 gene in the mouse genome. Detailed methodologies for homologous recombination in mice are available (see Capecchi, Science (1989) 244:1288-1292; Joyner et al., Nature (1989) 338:153-156). Procedures for the production of non-rodent transgenic mammals and other animals are also available (Houdebine and Chourrout, supra; Pursel et al., Science (1989) 244:1281-1288; Simms et al., Bio/Technology (1988) 6:179-183). In a preferred embodiment, knock-out animals, such as mice harboring a knockout of a specific gene, may be used to produce antibodies against the human counterpart of the gene that has been knocked out (Claesson M H et al., (1994) Scan J Immunol 40:257-264; Declerck P J et al., (1995) J Biol Chem. 270:8397-400).

In another embodiment, the transgenic animal is a “knock-in” animal having an alteration in its genome that results in altered expression (e.g., increased (including ectopic) or decreased expression) of the VIPR1 gene, e.g., by introduction of additional copies of VIPR1, or by operatively inserting a regulatory sequence that provides for altered expression of an endogenous copy of the VIPR1 gene. Such regulatory sequences include inducible, tissue-specific, and constitutive promoters and enhancer elements. The knock-in can be homozygous or heterozygous.

Transgenic nonhuman animals can also be produced that contain selected systems allowing for regulated expression of the transgene. One example of such a system that may be produced is the cre/loxP recombinase system of bacteriophage P1 (Lakso et al., PNAS (1992) 89:6232-6236; U.S. Pat. No. 4,959,317). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase. Another example of a recombinase system is the FLP recombinase system of Saccharomyces cerevisiae (O'Gorman et al. (1991) Science 251:1351-1355; U.S. Pat. No. 5,654,182). In a preferred embodiment, both Cre-LoxP and Flp-Frt are used in the same system to regulate expression of the transgene, and for sequential deletion of vector sequences in the same cell (Sun X et al (2000) Nat Genet 25:83-6).

The genetically modified animals can be used in genetic studies to further elucidate the E2F/RB pathway, as animal models of disease and disorders implicating defective E2F/RB function, and for in vivo testing of candidate therapeutic agents, such as those identified in screens described below. The candidate therapeutic agents are administered to a genetically modified animal having altered VIPR1 function and phenotypic changes are compared with appropriate control animals such as genetically modified animals that receive placebo treatment, and/or animals with unaltered VIPR1 expression that receive candidate therapeutic agent.

In addition to the above-described genetically modified animals having altered VIPR1 function, animal models having defective E2F/RB function (and otherwise normal VIPR1 function), can be used in the methods of the present invention. For example, a E2F/RB knockout mouse can be used to assess, in vivo, the activity of a candidate E2F/RB modulating agent identified in one of the in vitro assays described below. Preferably, the candidate E2F/RB modulating agent when administered to a model system with cells defective in E2F/RB function, produces a detectable phenotypic change in the model system indicating that the E2F/RB function is restored, i.e., the cells exhibit normal cell cycle progression.

Modulating Agents

The invention provides methods to identify agents that interact with and/or modulate the function of VIPR1 and/or the E2F/RB pathway. Modulating agents identified by the methods are also part of the invention. Such agents are useful in a variety of diagnostic and therapeutic applications associated with the E2F/RB pathway, as well as in further analysis of the VIPR1 protein and its contribution to the E2F/RB pathway. Accordingly, the invention also provides methods for modulating the E2F/RB pathway comprising the step of specifically modulating VIPR1 activity by administering an VIPR1-interacting or -modulating agent.

As used herein, a “VIPR1-modulating agent” is any agent that modulates VIPR1 function, for example, an agent that interacts with VIPR1 to inhibit or enhance VIPR1 activity or otherwise affect normal VIPR1 function. VIPR1 function can be affected at any level, including transcription, protein expression, protein localization, and cellular or extra-cellular activity. In a preferred embodiment, the VIPR1-modulating agent specifically modulates the function of the VIPR1. The phrases “specific modulating agent”, “specifically modulates”, etc., are used herein to refer to modulating agents that directly bind to the VIPR1 polypeptide or nucleic acid, and preferably inhibit, enhance, or otherwise alter, the function of the VIPR1. These phrases also encompass modulating agents that alter the interaction of the VIPR1 with a binding partner, substrate, or cofactor (e.g. by binding to a binding partner of a VIPR1, or to a protein/binding partner complex, and altering VIPR1 function). In a further preferred embodiment, the VIPR1-modulating agent is a modulator of the E2F/RB pathway (e.g. it restores and/or upregulates E2F/RB function) and thus is also an E2F/RB-modulating agent.

Preferred VIPR1-modulating agents include small molecule compounds; VIPR1-interacting proteins, including antibodies and other biotherapeutics; and nucleic acid modulators such as antisense and RNA inhibitors. The modulating agents may be formulated in pharmaceutical compositions, for example, as compositions that may comprise other active ingredients, as in combination therapy, and/or suitable carriers or excipients. Techniques for formulation and administration of the compounds may be found in “Remington's Pharmaceutical Sciences” Mack Publishing Co., Easton, Pa., 19^{th} edition.

Small Molecule Modulators

Small molecules are often preferred to modulate function of proteins with enzymatic function, and/or containing protein interaction domains. Chemical agents, referred to in the art as “small molecule” compounds are typically organic, non-peptide molecules, having a molecular weight up to 10,000, preferably up to 5,000, more preferably up to 1,000, and most preferably up to 500 daltons. This class of modulators includes chemically synthesized molecules, for instance, compounds from combinatorial chemical libraries. Synthetic compounds may be rationally designed or identified based on known or inferred properties of the VIPR1 protein or may be identified by screening compound libraries. Alternative appropriate modulators of this class are natural products, particularly secondary metabolites from organisms such as plants or fungi, which can also be identified by screening compound libraries for VIPR1-modulating activity. Methods for generating and obtaining compounds are well known in the art (Schreiber S L, Science (2000) 151: 1964-1969; Radmann J and Gunther J, Science (2000) 151:1947-1948).

Small molecule modulators identified from screening assays, as described below, can be used as lead compounds from which candidate clinical compounds may be designed, optimized, and synthesized. Such clinical compounds may have utility in treating pathologies associated with the E2F/RB pathway. The activity of candidate small molecule modulating agents may be improved several-fold through iterative secondary functional validation, as further described below, structure determination, and candidate modulator modification and testing. Additionally, candidate clinical compounds are generated with specific regard to clinical and pharmacological properties. For example, the reagents may be derivatized and re-screened using in vitro and in vivo assays to optimize activity and minimize toxicity for pharmaceutical development.

Protein Modulators

Specific VIPR1-interacting proteins are useful in a variety of diagnostic and therapeutic applications related to the E2F/RB pathway and related disorders, as well as in validation assays for other VIPR1-modulating agents. In a preferred embodiment, VIPR1-interacting proteins affect normal VIPR1 function, including transcription, protein expression, protein localization, and cellular or extra-cellular activity. In another embodiment, VIPR1-interacting proteins are useful in detecting and providing information about the function of VIPR1 proteins, as is relevant to E2F/RB related disorders, such as cancer (e.g., for diagnostic means).

A VIPR1-interacting protein may be endogenous, i.e. one that naturally interacts genetically or biochemically with an VIPR1, such as a member of the VIPR1 pathway that modulates VIPR1 expression, localization, and/or activity. VIPR1-modulators include dominant negative forms of VIPR1-interacting proteins and of VIPR1 proteins themselves. Yeast two-hybrid and variant screens offer preferred methods for identifying endogenous VIPR1-interacting proteins (Finley, R. L. et al. (1996) in DNA Cloning-Expression Systems: A Practical Approach, eds. Glover D. & Hames B. D (Oxford University Press, Oxford, England), pp. 169-203; Fashema S F et al., Gene (2000) 250:1-14; Drees B L Curr Opin Chem Biol (1999) 3:64-70; Vidal M and Legrain P Nucleic Acids Res (1999) 27:919-29; and U.S. Pat. No. 5,928,868). Mass spectrometry is an alternative preferred method for the elucidation of protein complexes (reviewed in, e.g., Pandley A and Mann M, Nature (2000) 405:837-846; Yates J R 3^{rd}, Trends Genet (2000) 16:5-8).

A VIPR1-interacting protein may be an exogenous protein, such as an VIPR1-specific antibody or a T-cell antigen receptor (see, e.g., Harlow and Lane (1988) Antibodies, A Laboratory Manual, Cold Spring Harbor Laboratory; Harlow and Lane (1999) Using antibodies: a laboratory manual. Cold Spring Harbor, N.Y.: Cold Spring Harbor Laboratory Press). VIPR1 antibodies are further discussed below.

In preferred embodiments, an VIPR1-interacting protein specifically binds an VIPR1 protein. In alternative preferred embodiments, an VIPR1-modulating agent binds an VIPR1 substrate, binding partner, or cofactor.

Antibodies

In another embodiment, the protein modulator is an VIPR1 specific antibody agonist or antagonist. The antibodies have therapeutic and diagnostic utilities, and can be used in screening assays to identify VIPR1 modulators. The antibodies can also be used in dissecting the portions of the VIPR1 pathway responsible for various cellular responses and in the general processing and maturation of the VIPR1.

Antibodies that specifically bind VIPR1 polypeptides can be generated using known methods. Preferably the antibody is specific to a mammalian ortholog of VIPR1 polypeptide, and more preferably, to human VIPR1. Antibodies may be polyclonal, monoclonal (mAbs), humanized or chimeric antibodies, single chain antibodies, Fab fragments, F(ab′).sub.2 fragments, fragments produced by a FAb expression library, anti-idiotypic (anti-Id) antibodies, and epitope-binding fragments of any of the above. Epitopes of VIPR1 which are particularly antigenic can be selected, for example, by routine screening of VIPR1 polypeptides for antigenicity or by applying a theoretical method for selecting antigenic regions of a protein (Hopp and Wood (1981), Proc. Natl. Acad. Sci. U.S.A. 78:3824-28; Hopp and Wood, (1983) Mol. Immunol. 20:483-89; Sutcliffe et al., (1983) Science 219:660-66) to the amino acid sequence of an VIPR1. Monoclonal antibodies with affinities of 10⁸ M⁻¹ preferably 10⁹ M⁻¹ to 10¹⁰ M⁻¹, or stronger can be made by standard procedures as described (Harlow and Lane, supra; Goding (1986) Monoclonal Antibodies: Principles and Practice (2d ed) Academic Press, New York; and U.S. Pat. Nos. 4,381,292; 4,451,570; and 4,618,577). Antibodies may be generated against crude cell extracts of VIPR1 or substantially purified fragments thereof. If VIPR1 fragments are used, they preferably comprise at least 10, and more preferably, at least 20 contiguous amino acids of an VIPR1 protein. In a particular embodiment, VIPR1-specific antigens and/or immunogens are coupled to carrier proteins that stimulate the immune response. For example, the subject polypeptides are covalently coupled to the keyhole limpet hemocyanin (KLH) carrier, and the conjugate is emulsified in Freund's complete adjuvant, which enhances the immune response. An appropriate immune system such as a laboratory rabbit or mouse is immunized according to conventional protocols.

The presence of VIPR1-specific antibodies is assayed by an appropriate assay such as a solid phase enzyme-linked immunosorbant assay (ELISA) using immobilized corresponding VIPR1 polypeptides. Other assays, such as radioimmunoassays or fluorescent assays might also be used.

Chimeric antibodies specific to VIPR1 polypeptides can be made that contain different portions from different animal species. For instance, a human immunoglobulin constant region may be linked to a variable region of a murine mAb, such that the antibody derives its biological activity from the human antibody, and its binding specificity from the murine fragment. Chimeric antibodies are produced by splicing together genes that encode the appropriate regions from each species (Morrison et al., Proc. Natl. Acad. Sci. (1984) 81:6851-6855; Neuberger et al., Nature (1984) 312:604-608; Takeda et al., Nature (1985) 31:452-454). Humanized antibodies, which are a form of chimeric antibodies, can be generated by grafting complementary-determining regions (CDRs) (Carlos, T. M., J. M. Harlan. 1994. Blood 84:2068-2101) of mouse antibodies into a background of human framework regions and constant regions by recombinant DNA technology (Riechmann LM, et al., 1988 Nature 323: 323-327). Humanized antibodies contain ˜10% murine sequences and ˜90% human sequences, and thus further reduce or eliminate immunogenicity, while retaining the antibody specificities (Co M S, and Queen C. 1991 Nature 351: 501-501; Morrison S L. 1992 Ann. Rev. Immun. 10:239-265). Humanized antibodies and methods of their production are well-known in the art (U.S. Pat. Nos. 5,530,101, 5,585,089, 5,693,762, and 6,180,370).

VIPR1-specific single chain antibodies which are recombinant, single chain polypeptides formed by linking the heavy and light chain fragments of the Fv regions via an amino acid bridge, can be produced by methods known in the art (U.S. Pat. No. 4,946,778; Bird, Science (1988) 242:423-426; Huston et al., Proc. Natl. Acad. Sci. USA (1988) 85:5879-5883; and Ward et al., Nature (1989) 334:544-546).

Other suitable techniques for antibody production involve in vitro exposure of lymphocytes to the antigenic polypeptides or alternatively to selection of libraries of antibodies in phage or similar vectors (Huse et al., Science (1989) 246:1275-1281). As used herein, T-cell antigen receptors are included within the scope of antibody modulators (Harlow and Lane, 1988, supra).

The polypeptides and antibodies of the present invention may be used with or without modification. Frequently, antibodies will be labeled by joining, either covalently or non-covalently, a substance that provides for a detectable signal, or that is toxic to cells that express the targeted protein (Menard S, et al., Int J. Biol Markers (1989) 4:131-134). A wide variety of labels and conjugation techniques are known and are reported extensively in both the scientific and patent literature. Suitable labels include radionuclides, enzymes, substrates, cofactors, inhibitors, fluorescent moieties, fluorescent emitting lanthanide metals, chemiluminescent moieties, bioluminescent moieties, magnetic particles, and the like (U.S. Pat. Nos. 3,817,837; 3,850,752; 3,939,350; 3,996,345; 4,277,437; 4,275,149; and 4,366,241). Also, recombinant immunoglobulins may be produced (U.S. Pat. No. 4,816,567). Antibodies to cytoplasmic polypeptides may be delivered and reach their targets by conjugation with membrane-penetrating toxin proteins (U.S. Pat. No. 6,086,900).

When used therapeutically in a patient, the antibodies of the subject invention are typically administered parenterally, when possible at the target site, or intravenously. The therapeutically effective dose and dosage regimen is determined by clinical studies. Typically, the amount of antibody administered is in the range of about 0.1 mg/kg—to about 10 mg/kg of patient weight. For parenteral administration, the antibodies are formulated in a unit dosage injectable form (e.g., solution, suspension, emulsion) in association with a pharmaceutically acceptable vehicle. Such vehicles are inherently nontoxic and non-therapeutic. Examples are water, saline, Ringer's solution, dextrose solution, and 5% human serum albumin. Nonaqueous vehicles such as fixed oils, ethyl oleate, or liposome carriers may also be used. The vehicle may contain minor amounts of additives, such as buffers and preservatives, which enhance isotonicity and chemical stability or otherwise enhance therapeutic potential. The antibodies' concentrations in such vehicles are typically in the range of about 1 mg/ml to about 10 mg/ml. Immunotherapeutic methods are further described in the literature (U.S. Pat. No. 5,859,206; WO0073469).

Specific Biotherapeutics

In a preferred embodiment, an VIPR1-interacting protein may have biotherapeutic applications. Biotherapeutic agents formulated in pharmaceutically acceptable carriers and dosages may be used to activate or inhibit signal transduction pathways. This modulation may be accomplished by binding a ligand, thus inhibiting the activity of the pathway; or by binding a receptor, either to inhibit activation of, or to activate, the receptor. Alternatively, the biotherapeutic may itself be a ligand capable of activating or inhibiting a receptor. Biotherapeutic agents and methods of producing them are described in detail in U.S. Pat. No. 6,146,628.

When the VIPR1 is a ligand, it may be used as a biotherapeutic agent to activate or inhibit its natural receptor. Alternatively, antibodies against VIPR1, as described in the previous section, may be used as biotherapeutic agents.

When the VIPR1 is a receptor, its ligand(s), antibodies to the ligand(s) or the VIPR1 itself may be used as biotherapeutics to modulate the activity of VIPR1 in the E2F/RB pathway.

Nucleic Acid Modulators

Other preferred VIPR1-modulating agents comprise nucleic acid molecules, such as antisense oligomers or double stranded RNA (dsRNA), which generally inhibit VIPR1 activity. Preferred nucleic acid modulators interfere with the function of the VIPR1 nucleic acid such as DNA replication, transcription, translocation of the VIPR1 RNA to the site of protein translation, translation of protein from the VIPR1 RNA, splicing of the VIPR1 RNA to yield one or more mRNA species, or catalytic activity which may be engaged in or facilitated by the VIPR1 RNA.

In one embodiment, the antisense oligomer is an oligonucleotide that is sufficiently complementary to a VIPR1 mRNA to bind to and prevent translation, preferably by binding to the 5′ untranslated region. VIPR1-specific antisense oligonucleotides, preferably range from at least 6 to about 200 nucleotides. In some embodiments the oligonucleotide is preferably at least 10, 15, or 20 nucleotides in length. In other embodiments, the oligonucleotide is preferably less than 50, 40, or 30 nucleotides in length. The oligonucleotide can be DNA or RNA or a chimeric mixture or derivatives or modified versions thereof, single-stranded or double-stranded. The oligonucleotide can be modified at the base moiety, sugar moiety, or phosphate backbone. The oligonucleotide may include other appending groups such as peptides, agents that facilitate transport across the cell membrane, hybridization-triggered cleavage agents, and intercalating agents.

In another embodiment, the antisense oligomer is a phosphothioate morpholino oligomer (PMO). PMOs are assembled from four different morpholino subunits, each of which contain one of four genetic bases (A, C, G, or T) linked to a six-membered morpholine ring. Polymers of these subunits are joined by non-ionic phosphodiamidate intersubunit linkages. Details of how to make and use PMOs and other antisense oligomers are well known in the art (e.g. see WO99/18193; Probst J C, Antisense Oligodeoxynucleotide and Ribozyme Design, Methods. (2000) 22(3):271-281; Summerton J, and Weller D. 1997 Antisense Nucleic Acid Drug Dev.: 7:187-95; U.S. Pat. No. 5,235,033; and U.S. Pat. No. 5,378,841).

Alternative preferred VIPR1 nucleic acid modulators are double-stranded RNA species mediating RNA interference (RNAi). RNAi is the process of sequence-specific, post-transcriptional gene silencing in animals and plants, initiated by double-stranded RNA (dsRNA) that is homologous in sequence to the silenced gene. Methods relating to the use of RNAi to silence genes in C. elegans, Drosophila, plants, and humans are known in the art (Fire A, et al., 1998 Nature 391:806-811; Fire, A. Trends Genet. 15, 358-363 (1999); Sharp, P. A. RNA interference 2001. Genes Dev. 15, 485-490 (2001); Hammond, S. M., et al., Nature Rev. Genet. 2, 110-1119 (2001); Tuschl, T. Chem. Biochem. 2, 239-245 (2001); Hamilton, A. et al., Science 286, 950-952 (1999); Hammond, S. M., et al., Nature 404, 293-296 (2000); Zamore, P. D., et al., Cell 101, 25-33 (2000); Bernstein, E., et al., Nature 409, 363-366 (2001); Elbashir, S. M., et al., Genes Dev. 15, 188-200 (2001); WO0129058; WO9932619; Elbashir S M, et al., 2001 Nature 411:494-498; Novina CD and Sharp P. Nature 430:161-164; Soutschek J et al 2004 Nature 432:173-178; Hsieh AC et al. (2004) NAR 32(3):893-901). Examples of siRNA that modulate VIPR1 expression are shown in SEQ ID NOs: 3-10.

Nucleic acid modulators are commonly used as research reagents, diagnostics, and therapeutics. For example, antisense oligonucleotides, which are able to inhibit gene expression with exquisite specificity, are often used to elucidate the function of particular genes (see, for example, U.S. Pat. No. 6,165,790). Nucleic acid modulators are also used, for example, to distinguish between functions of various members of a biological pathway. For example, antisense oligomers have been employed as therapeutic moieties in the treatment of disease states in animals and man and have been demonstrated in numerous clinical trials to be safe and effective (Milligan J F, et al, Current Concepts in Antisense Drug Design, J Med Chem. (1993) 36:1923-1937; Tonkinson J L et al., Antisense Oligodeoxynucleotides as Clinical Therapeutic Agents, Cancer Invest. (1996) 14:54-65). Accordingly, in one aspect of the invention, an VIPR1-specific nucleic acid modulator is used in an assay to further elucidate the role of the VIPR1 in the E2F/RB pathway, and/or its relationship to other members of the pathway. In another aspect of the invention, an VIPR1-specific antisense oligomer is used as a therapeutic agent for treatment of E2F/RB-related disease states.

Assay Systems

The invention provides assay systems and screening methods for identifying specific modulators of VIPR1 activity. As used herein, an “assay system” encompasses all the components required for performing and analyzing results of an assay that detects and/or measures a particular event. In general, primary assays are used to identify or confirm a modulator's specific biochemical or molecular effect with respect to the VIPR1 nucleic acid or protein. In general, secondary assays further assess the activity of an VIPR1 modulating agent identified by a primary assay and may confirm that the modulating agent affects VIPR1 in a manner relevant to the E2F/RB pathway. In some cases, VIPR1 modulators will be directly tested in a secondary assay.

In a preferred embodiment, the screening method comprises contacting a suitable assay system comprising an VIPR1 polypeptide or nucleic acid with a candidate agent under conditions whereby, but for the presence of the agent, the system provides a reference activity (e.g. binding activity), which is based on the particular molecular event the screening method detects. A statistically significant difference between the agent-biased activity and the reference activity indicates that the candidate agent modulates VIPR1 activity, and hence the E2F/RB pathway. The VIPR1 polypeptide or nucleic acid used in the assay may comprise any of the nucleic acids or polypeptides described above.

Primary Assays

The type of modulator tested generally determines the type of primary assay.

Primary Assays for Small Molecule Modulators

For small molecule modulators, screening assays are used to identify candidate modulators. Screening assays may be cell-based or may use a cell-free system that recreates or retains the relevant biochemical reaction of the target protein (reviewed in Sittampalam G S et al., Curr Opin Chem Biol (1997) 1:384-91 and accompanying references). As used herein the term “cell-based” refers to assays using live cells, dead cells, or a particular cellular fraction, such as a membrane, endoplasmic reticulum, or mitochondrial fraction. The term “cell free” encompasses assays using substantially purified protein (either endogenous or recombinantly produced), partially purified or crude cellular extracts. Screening assays may detect a variety of molecular events, including protein-DNA interactions, protein-protein interactions (e.g., receptor-ligand binding), transcriptional activity (e.g., using a reporter gene), enzymatic activity (e.g., via a property of the substrate), activity of second messengers, immunogenicty and changes in cellular morphology or other cellular characteristics. Appropriate screening assays may use a wide range of detection methods including fluorescent, radioactive, colorimetric, spectrophotometric, and amperometric methods, to provide a read-out for the particular molecular event detected.

Cell-based screening assays usually require systems for recombinant expression of VIPR1 and any auxiliary proteins demanded by the particular assay. Appropriate methods for generating recombinant proteins produce sufficient quantities of proteins that retain their relevant biological activities and are of sufficient purity to optimize activity and assure assay reproducibility. Yeast two-hybrid and variant screens, and mass spectrometry provide preferred methods for determining protein-protein interactions and elucidation of protein complexes. In certain applications, when VIPR1-interacting proteins are used in screens to identify small molecule modulators, the binding specificity of the interacting protein to the VIPR1 protein may be assayed by various known methods such as substrate processing (e.g. ability of the candidate VIPR1-specific binding agents to function as negative effectors in VIPR1-expressing cells), binding equilibrium constants (usually at least about 10⁷ M⁻¹, preferably at least about 10⁸ M⁻¹, more preferably at least about 10⁹ M⁻¹), and immunogenicity (e.g. ability to elicit VIPR1 specific antibody in a heterologous host such as a mouse, rat, goat or rabbit). For enzymes and receptors, binding may be assayed by, respectively, substrate and ligand processing.

The screening assay may measure a candidate agent's ability to specifically bind to or modulate activity of an VIPR1 polypeptide, a fusion protein thereof, or to cells or membranes bearing the polypeptide or fusion protein. The VIPR1 polypeptide can be full length or a fragment thereof that retains functional VIPR1 activity. The VIPR1 polypeptide may be fused to another polypeptide, such as a peptide tag for detection or anchoring, or to another tag. The VIPR1 polypeptide is preferably human VIPR1, or is an ortholog or derivative thereof as described above. In a preferred embodiment, the screening assay detects candidate agent-based modulation of VIPR1 interaction with a binding target, such as an endogenous or exogenous protein or other substrate that has VIPR1—specific binding activity, and can be used to assess normal VIPR1 gene function.

Suitable assay formats that may be adapted to screen for VIPR1 modulators are known in the art. Preferred screening assays are high throughput or ultra high throughput and thus provide automated, cost-effective means of screening compound libraries for lead compounds (Fernandes P B, Curr Opin Chem Biol (1998) 2:597-603; Sundberg S A, Curr Opin Biotechnol 2000, 11:47-53). In one preferred embodiment, screening assays uses fluorescence technologies, including fluorescence polarization, time-resolved fluorescence, and fluorescence resonance energy transfer. These systems offer means to monitor protein-protein or DNA-protein interactions in which the intensity of the signal emitted from dye-labeled molecules depends upon their interactions with partner molecules (e.g., Selvin P R, Nat Struct Biol (2000) 7:730-4; Fernandes P B, supra; Hertzberg R P and Pope A J, Curr Opin Chem Biol (2000) 4:445-451).

A variety of suitable assay systems may be used to identify candidate VIPR1 and E2F/RB pathway modulators (e.g. U.S. Pat. No. 6,165,992 and U.S. Pat. No. 6,720,162 (kinase assays); U.S. Pat. Nos. 5,550,019 and 6,133,437 (apoptosis assays); WO 01/25487 (Helicase assays), U.S. Pat. No. 6,114,132 and U.S. Pat. No. 6,720,162 (phosphatase and protease assays), U.S. Pat. Nos. 5,976,782, 6,225,118 and 6,444,434 (angiogenesis assays), among others). Specific preferred assays are described in more detail below.

Protein kinases, key signal transduction proteins that may be either membrane-associated or intracellular, catalyze the transfer of gamma phosphate from adenosine triphosphate (ATP) to a serine, threonine or tyrosine residue in a protein substrate. Radioassays, which monitor the transfer from [gamma-³²P or -³³P]ATP, are frequently used to assay kinase activity. For instance, a scintillation assay for p56 (lck) kinase activity monitors the transfer of the gamma phosphate from [gamma-³³P] ATP to a biotinylated peptide substrate. The substrate is captured on a streptavidin coated bead that transmits the signal (Beveridge M et al., J Biomol Screen (2000) 5:205-212). This assay uses the scintillation proximity assay (SPA), in which only radio-ligand bound to receptors tethered to the surface of an SPA bead are detected by the scintillant immobilized within it, allowing binding to be measured without separation of bound from free ligand. Other assays for protein kinase activity may use antibodies that specifically recognize phosphorylated substrates. For instance, the kinase receptor activation (KIRA) assay measures receptor tyrosine kinase activity by ligand stimulating the intact receptor in cultured cells, then capturing solubilized receptor with specific antibodies and quantifying phosphorylation via phosphotyrosine ELISA (Sadick M D, Dev Biol Stand (1999) 97:121-133). Another example of antibody based assays for protein kinase activity is TRF (time-resolved fluorometry). This method utilizes europium chelate-labeled anti-phosphotyrosine antibodies to detect phosphate transfer to a polymeric substrate coated onto microtiter plate wells. The amount of phosphorylation is then detected using time-resolved, dissociation-enhanced fluorescence (Braunwalder A F, et al., Anal Biochem 1996 Jul. 1; 238(2):159-64). Yet other assays for kinases involve uncoupled, pH sensitive assays that can be used for high-throughput screening of potential inhibitors or for determining substrate specificity. Since kinases catalyze the transfer of a gamma-phosphoryl group from ATP to an appropriate hydroxyl acceptor with the release of a proton, a pH sensitive assay is based on the detection of this proton using an appropriately matched buffer/indicator system (Chapman E and Wong C H (2002) Bioorg Med Chem. 10:551-5).

Protein phosophatases catalyze the removal of a gamma phosphate from a serine, threonine or tyrosine residue in a protein substrate. Since phosphatases act in opposition to kinases, appropriate assays measure the same parameters as kinase assays. In one example, the dephosphorylation of a fluorescently labeled peptide substrate allows trypsin cleavage of the substrate, which in turn renders the cleaved substrate significantly more fluorescent (Nishikata M et al., Biochem J (1999) 343:35-391). In another example, fluorescence polarization (FP), a solution-based, homogeneous technique requiring no immobilization or separation of reaction components, is used to develop high throughput screening (HTS) assays for protein phosphatases. This assay uses direct binding of the phosphatase with the target, and increasing concentrations of target-phosphatase increase the rate of dephosphorylation, leading to a change in polarization (Parker G J et al., (2000) J Biomol Screen 5:77-88).

Proteases are enzymes that cleave protein substrates at specific sites. Exemplary assays detect the alterations in the spectral properties of an artificial substrate that occur upon protease-mediated cleavage. In one example, synthetic caspase substrates containing four amino acid proteolysis recognition sequences, separating two different fluorescent tags are employed; fluorescence resonance energy transfer detects the proximity of these fluorophores, which indicates whether the substrate is cleaved (Mahajan NP et al., Chem Biol (1999) 6:401-409).

Helicases are involved in unwinding double stranded DNA and RNA. In one embodiment, an assay for DNA helicase activity detects the displacement of a radio-labeled oligonucleotide from single stranded DNA upon initiation of unwinding (Sivaraja M et al., Anal Biochem (1998) 265:22-27). An assay for RNA helicase activity uses the scintillation proximity (SPA) assay to detect the displacement of a radio-labeled oligonucleotide from single stranded RNA (Kyono K et al., Anal Biochem (1998) 257:120-126).

Peptidyl-prolyl isomerase (PPIase) proteins, which include cyclophilins, FK506 binding proteins and paravulins, catalyze the isomerization of cis-trans proline peptide bonds in oligopeptides and are thought to be essential for protein folding during protein synthesis in the cell. Spectrophotometric assays for PPIase activity can detect isomerization of labeled peptide substrates, either by direct measurement of isomer-specific absorbance, or by coupling isomerization to isomer-specific cleavage by chymotrypsin (Scholz C et al., FEBS Lett (1997) 414:69-73; Janowski B et al., Anal Biochem (1997) 252:299-307; Kullertz G et al., Clin Chem (1998) 44:502-8). Alternative assays use the scintillation proximity or fluorescence polarization assay to screen for ligands of specific PPIases (Graziani F et al., J Biolmol Screen (1999) 4:3-7; Dubowchik G M et al., Bioorg Med Chem Lett (2000) 10:559-562). Assays for 3,2-trans-enoyl-CoA isomerase activity have also been described (Binstock, J. F., and Schulz, H. (1981) Methods Enzymol. 71:403-411; Geisbrecht B V et al (1999) J Biol Chem. 274:21797-803). These assays use 3-cis-octenoyl-CoA as a substrate, and reaction progress is monitored spectrophotometrically using a coupled assay for the isomerization of 3-cis-octenoyl-CoA to 2-trans-octenoyl-CoA.

Ubiquitination is a process of attaching ubiquitin to a protein prior to the selective proteolysis of that protein in the cell. Assays based on fluorescence resonance energy transfer to screen for ubiquitination inhibitors are known in the art (Boisclair M D et al., J Biomol Screen 2000 5:319-328).

Hydrolases catalyze the hydrolysis of a substrate such as esterases, lipases, peptidases, nucleotidases, and phosphatases, among others. Enzyme activity assays may be used to measure hydrolase activity. The activity of the enzyme is determined in presence of excess substrate, by spectrophotometrically measuring the rate of appearance of reaction products. High throughput arrays and assays for hydrolases are known to those skilled in the art (Park C B and Clark D S (2002) Biotech Bioeng 78:229-235).

Kinesins are motor proteins. Assays for kinesins involve their ATPase activity, such as described in Blackburn et al (Blackburn C L, et al., (1999) J Org Chem 64:5565-5570). The ATPase assay is performed using the EnzCheck ATPase kit (Molecular Probes). The assays are performed using an Ultraspec spectrophotometer (Pharmacia), and the progress of the reaction are monitored by absorbance increase at 360 nm. Microtubules (1.7 mM final), kinesin (0.11 mM final), inhibitor (or DMSO blank at 5% final), and the EnzCheck components (purine nucleotide phosphorylase and MESG substrate) are premixed in the cuvette in a reaction buffer (40 mM PIPES pH 6.8, 5 mM paclitaxel, 1 mM EGTA, 5 mM MgCl2). The reaction is initiated by addition of MgATP (1 mM final).

High-throughput assays, such as scintillation proximity assays, for synthase enzymes involved in fatty acid synthesis are known in the art (He X et al (2000) Anal Biochem 2000 Jun. 15; 282(1):107-14).

Apoptosis assays. Apoptosis or programmed cell death is a suicide program is activated within the cell, leading to fragmentation of DNA, shrinkage of the cytoplasm, membrane changes and cell death. Apoptosis is mediated by proteolytic enzymes of the caspase family. Many of the altering parameters of a cell are measurable during apoptosis. Assays for apoptosis may be performed by terminal deoxynucleotidyl transferase-mediated digoxigenin-11-dUTP nick end labeling (TUNEL) assay. The TUNEL assay is used to measure nuclear DNA fragmentation characteristic of apoptosis (Lazebnik et al., 1994, Nature 371, 346), by following the incorporation of fluorescein-dUTP (Yonehara et al., 1989, J. Exp. Med. 169, 1747). Apoptosis may further be assayed by acridine orange staining of tissue culture cells (Lucas, R., et al., 1998, Blood 15:4730-41). Other cell-based apoptosis assays include the caspase-3/7 assay and the cell death nucleosome ELISA assay. The caspase 3/7 assay is based on the activation of the caspase cleavage activity as part of a cascade of events that occur during programmed cell death in many apoptotic pathways. In the caspase 3/7 assay (commercially available Apo-ONE™ Homogeneous Caspase-3/7 assay from Promega, cat #67790), lysis buffer and caspase substrate are mixed and added to cells. The caspase substrate becomes fluorescent when cleaved by active caspase 3/7. The nucleosome ELISA assay is a general cell death assay known to those skilled in the art, and available commercially (Roche, Cat #1774425). This assay is a quantitative sandwich-enzyme-immunoassay which uses monoclonal antibodies directed against DNA and histones respectively, thus specifically determining amount of mono- and oligonucleosomes in the cytoplasmic fraction of cell lysates. Mono and oligonucleosomes are enriched in the cytoplasm during apoptosis due to the fact that DNA fragmentation occurs several hours before the plasma membrane breaks down, allowing for accumulation in the cytoplasm. Nucleosomes are not present in the cytoplasmic fraction of cells that are not undergoing apoptosis. The Phospho-histone H2B assay is another apoptosis assay, based on phosphorylation of histone H2B as a result of apoptosis. Fluorescent dyes that are associated with phosphohistone H2B may be used to measure the increase of phosphohistone H2B as a result of apoptosis. Apoptosis assays that simultaneously measure multiple parameters associated with apoptosis have also been developed. In such assays, various cellular parameters that can be associated with antibodies or fluorescent dyes, and that mark various stages of apoptosis are labeled, and the results are measured using instruments such as Cellomics™ ArrayScan® HCS System. The measurable parameters and their markers include anti-active caspase-3 antibody which marks intermediate stage apoptosis, anti-PARP-p85 antibody (cleaved PARP) which marks late stage apoptosis, Hoechst labels which label the nucleus and are used to measure nuclear swelling as a measure of early apoptosis and nuclear condensation as a measure of late apoptosis, TOTO-3 fluorescent dye which labels DNA of dead cells with high cell membrane permeability, and anti-alpha-tubulin or F-actin labels, which assess cytoskeletal changes in cells and correlate well with TOTO-3 label. These assays may also be used for involvement of a gene in cell cycle and assessment of alterations in cell morphology.

An apoptosis assay system may comprise a cell that expresses a VIPR1, and that optionally has defective E2F/RB function (e.g. E2F/RB is over-expressed or under-expressed relative to wild-type cells). A test agent can be added to the apoptosis assay system and changes in induction of apoptosis relative to controls where no test agent is added, identify candidate E2F/RB modulating agents. In some embodiments of the invention, an apoptosis assay may be used as a secondary assay to test a candidate E2F/RB modulating agents that is initially identified using a cell-free assay system. An apoptosis assay may also be used to test whether VIPR1 function plays a direct role in apoptosis. For example, an apoptosis assay may be performed on cells that over- or under-express VIPR1 relative to wild type cells. Differences in apoptotic response compared to wild type cells suggests that the VIPR1 plays a direct role in the apoptotic response. Apoptosis assays are described further in U.S. Pat. No. 6,133,437.

Cell proliferation and cell cycle assays. Cell proliferation may be assayed via bromodeoxyuridine (BRDU) incorporation. This assay identifies a cell population undergoing DNA synthesis by incorporation of BRDU into newly-synthesized DNA. Newly-synthesized DNA may then be detected using an anti-BRDU antibody (Hoshino et al., 1986, Int. J. Cancer 38, 369; Campana et al., 1988, J. Immunol. Meth. 107, 79), or by other means.

Cell proliferation is also assayed via phospho-histone H3 staining, which identifies a cell population undergoing mitosis by phosphorylation of histone H3. Phosphorylation of histone H3 at serine 10 is detected using an antibody specific to the phosphorylated form of the serine 10 residue of histone H3. (Chadlee, D. N. 1995, J. Biol. Chem 270:20098-105). Cell Proliferation may also be examined using [³H]-thymidine incorporation (Chen, J., 1996, Oncogene 13:1395-403; Jeoung, J., 1995, J. Biol. Chem. 270:18367-73). This assay allows for quantitative characterization of S-phase DNA syntheses. In this assay, cells synthesizing DNA will incorporate [³H]-thymidine into newly synthesized DNA. Incorporation can then be measured by standard techniques such as by counting of radioisotope in a scintillation counter (e.g., Beckman LS 3800 Liquid Scintillation Counter). Another proliferation assay uses the dye Alamar Blue (available from Biosource International), which fluoresces when reduced in living cells and provides an indirect measurement of cell number (Voytik-Harbin S L et al., 1998, In Vitro Cell Dev Biol Anim 34:239-46). Yet another proliferation assay, the MTS assay, is based on in vitro cytotoxicity assessment of industrial chemicals, and uses the soluble tetrazolium salt, MTS. MTS assays are commercially available, for example, the Promega CellTiter 96® AQueous Non-Radioactive Cell Proliferation Assay (Cat. #G5421).

Cell proliferation may also be assayed by colony formation in soft agar, or clonogenic survival assay (Sambrook et al., Molecular Cloning, Cold Spring Harbor (1989)). For example, cells transformed with VIPR1 are seeded in soft agar plates, and colonies are measured and counted after two weeks incubation.

Cell proliferation may also be assayed by measuring ATP levels as indicator of metabolically active cells. Such assays are commercially available, for example Cell Titer-Glo™, which is a luminescent homogeneous assay available from Promega.

Involvement of a gene in the cell cycle may be assayed by flow cytometry (Gray J W et al. (1986) Int J Radiat Biol Relat Stud Phys Chem Med 49:237-55). Cells transfected with an VIPR1 may be stained with propidium iodide and evaluated in a flow cytometer (available from Becton Dickinson), which indicates accumulation of cells in different stages of the cell cycle.

Involvement of a gene in the cell cycle, cell movement, or cell morphology may further be assessed using the Cellomics™ ArrayScan® HCS System, as described above. For cell motility, cells are seeded in 96 well plates, then treated with modulator of interest, such as RNAi, then transferred to collagen plates containing fluorescent microspheres. Replated cells are later fixed and stained with rhodamine-Alexa546, and motility tracks are viewed and measured using the HCS system.

Accordingly, a cell proliferation, cell movement, cell morphology, or cell cycle assay system may comprise a cell that expresses a VIPR1, and that optionally has defective E2F/RB function (e.g. E2F/RB is over-expressed or under-expressed relative to wild-type cells). A test agent can be added to the assay system and changes in cell proliferation or cell cycle relative to controls where no test agent is added, identify candidate E2F/RB modulating agents. In some embodiments of the invention, the cell proliferation or cell cycle assay may be used as a secondary assay to test a candidate E2F/RB modulating agents that is initially identified using another assay system such as a cell-free assay system. A cell proliferation assay may also be used to test whether VIPR1 function plays a direct role in cell proliferation or cell cycle. For example, a cell proliferation or cell cycle assay may be performed on cells that over- or under-express VIPR1 relative to wild type cells. Differences in proliferation or cell cycle compared to wild type cells suggests that the VIPR1 plays a direct role in cell proliferation or cell cycle.

Angiogenesis. Angiogenesis may be assayed using various human endothelial cell systems, such as umbilical vein, coronary artery, or dermal cells. Suitable assays include Alamar Blue based assays (available from Biosource International) to measure proliferation; migration assays using fluorescent molecules, such as the use of Becton Dickinson Falcon HTS FluoroBlock cell culture inserts to measure migration of cells through membranes in presence or absence of angiogenesis enhancer or suppressors; and tubule formation assays based on the formation of tubular structures by endothelial cells on Matrigel® (Becton Dickinson). Accordingly, an angiogenesis assay system may comprise a cell that expresses an VIPR1, and that optionally has defective E2F/RB function (e.g. E2F/RB is over-expressed or under-expressed relative to wild-type cells). A test agent can be added to the angiogenesis assay system and changes in angiogenesis relative to controls where no test agent is added, identify candidate E2F/RB modulating agents. In some embodiments of the invention, the angiogenesis assay may be used as a secondary assay to test a candidate E2F/RB modulating agents that is initially identified using another assay system. An angiogenesis assay may also be used to test whether VIPR1 function plays a direct role in cell proliferation. For example, an angiogenesis assay may be performed on cells that over- or under-express VIPR1 relative to wild type cells. Differences in angiogenesis compared to wild type cells suggests that the VIPR1 plays a direct role in angiogenesis. U.S. Pat. Nos. 5,976,782, 6,225,118 and 6,444,434, among others, describe various angiogenesis assays.

Hypoxic induction. The alpha subunit of the transcription factor, hypoxia inducible factor-1 (HIF-1), is upregulated in tumor cells following exposure to hypoxia in vitro. Under hypoxic conditions, HIF-1 stimulates the expression of genes known to be important in tumour cell survival, such as those encoding glyolytic enzymes and VEGF. Induction of such genes by hypoxic conditions may be assayed by growing cells transfected with VIPR1 in hypoxic conditions (such as with 0.1% O2, 5% CO2, and balance N2, generated in a Napco 7001 incubator (Precision Scientific)) and normoxic conditions, followed by assessment of gene activity or expression by Taqman®. For example, a hypoxic induction assay system may comprise a cell that expresses a VIPR1, and that optionally has defective E2F/RB function (e.g. E2F/RB is over-expressed or under-expressed relative to wild-type cells). A test agent can be added to the hypoxic induction assay system and changes in hypoxic response relative to controls where no test agent is added, identify candidate E2F/RB modulating agents. In some embodiments of the invention, the hypoxic induction assay may be used as a secondary assay to test a candidate E2F/RB modulating agents that is initially identified using another assay system. A hypoxic induction assay may also be used to test whether VIPR1 function plays a direct role in the hypoxic response. For example, a hypoxic induction assay may be performed on cells that over- or under-express VIPR1 relative to wild type cells. Differences in hypoxic response compared to wild type cells suggests that the VIPR1 plays a direct role in hypoxic induction.

Cell adhesion. Cell adhesion assays measure adhesion of cells to purified adhesion proteins, or adhesion of cells to each other, in presence or absence of candidate modulating agents. Cell-protein adhesion assays measure the ability of agents to modulate the adhesion of cells to purified proteins. For example, recombinant proteins are produced, diluted to 2.5 g/mL in PBS, and used to coat the wells of a microtiter plate. The wells used for negative control are not coated. Coated wells are then washed, blocked with 1% BSA, and washed again. Compounds are diluted to 2× final test concentration and added to the blocked, coated wells. Cells are then added to the wells, and the unbound cells are washed off. Retained cells are labeled directly on the plate by adding a membrane-permeable fluorescent dye, such as calcein-AM, and the signal is quantified in a fluorescent microplate reader.

Cell-cell adhesion assays measure the ability of agents to modulate binding of cell adhesion proteins with their native ligands. These assays use cells that naturally or recombinantly express the adhesion protein of choice. In an exemplary assay, cells expressing the cell adhesion protein are plated in wells of a multiwell plate. Cells expressing the ligand are labeled with a membrane-permeable fluorescent dye, such as BCECF, and allowed to adhere to the monolayers in the presence of candidate agents. Unbound cells are washed off, and bound cells are detected using a fluorescence plate reader.

High-throughput cell adhesion assays have also been described. In one such assay, small molecule ligands and peptides are bound to the surface of microscope slides using a microarray spotter, intact cells are then contacted with the slides, and unbound cells are washed off. In this assay, not only the binding specificity of the peptides and modulators against cell lines are determined, but also the functional cell signaling of attached cells using immunofluorescence techniques in situ on the microchip is measured (Falsey J R et al., Bioconjug Chem. 2001 May-June; 12(3):346-53).

### Primary Assays for Antibody Modulators

For antibody modulators, appropriate primary assays test is a binding assay that tests the antibody's affinity to and specificity for the VIPR1 protein. Methods for testing antibody affinity and specificity are well known in the art (Harlow and Lane, 1988, 1999, supra). The enzyme-linked immunosorbant assay (ELISA) is a preferred method for detecting VIPR1-specific antibodies; others include FACS assays, radioimmunoassays, and fluorescent assays.

In some cases, screening assays described for small molecule modulators may also be used to test antibody modulators.

### Primary Assays for Nucleic Acid Modulators

For nucleic acid modulators, primary assays may test the ability of the nucleic acid modulator to inhibit or enhance VIPR1 gene expression, preferably mRNA expression. In general, expression analysis comprises comparing VIPR1 expression in like populations of cells (e.g., two pools of cells that endogenously or recombinantly express VIPR1) in the presence and absence of the nucleic acid modulator. Methods for analyzing mRNA and protein expression are well known in the art. For instance, Northern blotting, slot blotting, ribonuclease protection, quantitative RT-PCR (e.g., using the TaqMan®, PE Applied Biosystems), or microarray analysis may be used to confirm that VIPR1 mRNA expression is reduced in cells treated with the nucleic acid modulator (e.g., Current Protocols in Molecular Biology (1994) Ausubel F M et al., eds., John Wiley & Sons, Inc., chapter 4; Freeman W M et al., Biotechniques (1999) 26:112-125; Kallioniemi O P, Ann Med 2001, 33:142-147; Blohm D H and Guiseppi-Elie, A Curr Opin Biotechnol 2001, 12:41-47). Protein expression may also be monitored. Proteins are most commonly detected with specific antibodies or antisera directed against either the VIPR1 protein or specific peptides. A variety of means including Western blotting, ELISA, or in situ detection, are available (Harlow E and Lane D, 1988 and 1999, supra).

In some cases, screening assays described for small molecule modulators, particularly in assay systems that involve VIPR1 mRNA expression, may also be used to test nucleic acid modulators.

### Secondary Assays

Secondary assays may be used to further assess the activity of VIPR1-modulating agent identified by any of the above methods to confirm that the modulating agent affects VIPR1 in a manner relevant to the E2F/RB pathway. As used herein, VIPR1-modulating agents encompass candidate clinical compounds or other agents derived from previously identified modulating agent. Secondary assays can also be used to test the activity of a modulating agent on a particular genetic or biochemical pathway or to test the specificity of the modulating agent's interaction with VIPR1.

Secondary assays generally compare like populations of cells or animals (e.g., two pools of cells or animals that endogenously or recombinantly express VIPR1) in the presence and absence of the candidate modulator. In general, such assays test whether treatment of cells or animals with a candidate VIPR1-modulating agent results in changes in the E2F/RB pathway in comparison to untreated (or mock- or placebo-treated) cells or animals. Certain assays use “sensitized genetic backgrounds”, which, as used herein, describe cells or animals engineered for altered expression of genes in the E2F/RB or interacting pathways.

### Cell-Based Assays

Cell based assays may detect endogenous E2F/RB pathway activity or may rely on recombinant expression of E2F/RB pathway components. Any of the aforementioned assays may be used in this cell-based format. Candidate modulators are typically added to the cell media but may also be injected into cells or delivered by any other efficacious means.

### Animal Assays

A variety of non-human animal models of normal or defective E2F/RB pathway may be used to test candidate VIPR1 modulators. Models for defective E2F/RB pathway typically use genetically modified animals that have been engineered to mis-express (e.g., over-express or lack expression in) genes involved in the E2F/RB pathway. Assays generally require systemic delivery of the candidate modulators, such as by oral administration, injection, etc.

In a preferred embodiment, E2F/RB pathway activity is assessed by monitoring neovascularization and angiogenesis. Animal models with defective and normal E2F/RB are used to test the candidate modulator's affect on VIPR1 in Matrigel® assays. Matrigel® is an extract of basement membrane proteins, and is composed primarily of laminin, collagen IV, and heparin sulfate proteoglycan. It is provided as a sterile liquid at 4° C., but rapidly forms a solid gel at 37° C. Liquid Matrigel® is mixed with various angiogenic agents, such as bFGF and VEGF, or with human tumor cells which over-express the VIPR1. The mixture is then injected subcutaneously (SC) into female athymic nude mice (Taconic, Germantown, N.Y.) to support an intense vascular response. Mice with Matrigel® pellets may be dosed via oral (PO), intraperitoneal (IP), or intravenous (IV) routes with the candidate modulator. Mice are euthanized 5-12 days post-injection, and the Matrigel® pellet is harvested for hemoglobin analysis (Sigma plasma hemoglobin kit). Hemoglobin content of the gel is found to correlate the degree of neovascularization in the gel.

In another preferred embodiment, the effect of the candidate modulator on VIPR1 is assessed via tumorigenicity assays. Tumor xenograft assays are known in the art (see, e.g., Ogawa K et al., 2000, Oncogene 19:6043-6052). Xenografts are typically implanted SC into female athymic mice, 6-7 week old, as single cell suspensions either from a pre-existing tumor or from in vitro culture. The tumors which express the VIPR1 endogenously are injected in the flank, 1×10⁵ to 1×10⁷ cellsper mouse in a volume of 100 μL using a 27 gauge needle. Mice are then ear tagged and tumors are measured twice weekly. Candidate modulator treatment is initiated on the day the mean tumor weight reaches 100 mg. Candidate modulator is delivered IV, SC, IP, or PO by bolus administration. Depending upon the pharmacokinetics of each unique candidate modulator, dosing can be performed multiple times per day. The tumor weight is assessed by measuring perpendicular diameters with a caliper and calculated by multiplying the measurements of diameters in two dimensions. At the end of the experiment, the excised tumors maybe utilized for biomarker identification or further analyses. For immunohistochemistry staining, xenograft tumors are fixed in 4% paraformaldehyde, 0.1M phosphate, pH 7.2, for 6 hours at 4° C., immersed in 30% sucrose in PBS, and rapidly frozen in isopentane cooled with liquid nitrogen.

In another preferred embodiment, tumorogenicity is monitored using a hollow fiber assay, which is described in U.S. Pat. No. 5,698,413. Briefly, the method comprises implanting into a laboratory animal a biocompatible, semi-permeable encapsulation device containing target cells, treating the laboratory animal with a candidate modulating agent, and evaluating the target cells for reaction to the candidate modulator. Implanted cells are generally human cells from a pre-existing tumor or a tumor cell line. After an appropriate period of time, generally around six days, the implanted samples are harvested for evaluation of the candidate modulator. Tumorogenicity and modulator efficacy may be evaluated by assaying the quantity of viable cells present in the macrocapsule, which can be determined by tests known in the art, for example, MTT dye conversion assay, neutral red dye uptake, trypan blue staining, viable cell counts, the number of colonies formed in soft agar, the capacity of the cells to recover and replicate in vitro, etc.

In another preferred embodiment, a tumorogenicity assay use a transgenic animal, usually a mouse, carrying a dominant oncogene or tumor suppressor gene knockout under the control of tissue specific regulatory sequences; these assays are generally referred to as transgenic tumor assays. In a preferred application, tumor development in the transgenic model is well characterized or is controlled. In an exemplary model, the “RIP1-Tag2” transgene, comprising the SV40 large T-antigen oncogene under control of the insulin gene regulatory regions is expressed in pancreatic beta cells and results in islet cell carcinomas (Hanahan D, 1985, Nature 315:115-122; Parangi S et al, 1996, Proc Natl Acad Sci USA 93: 2002-2007; Bergers G et al, 1999, Science 284:808-812). An “angiogenic switch,” occurs at approximately five weeks, as normally quiescent capillaries in a subset of hyperproliferative islets become angiogenic. The RIP1-TAG2 mice die by age 14 weeks. Candidate modulators may be administered at a variety of stages, including just prior to the angiogenic switch (e.g., for a model of tumor prevention), during the growth of small tumors (e.g., for a model of intervention), or during the growth of large and/or invasive tumors (e.g., for a model of regression). Tumorogenicity and modulator efficacy can be evaluating life-span extension and/or tumor characteristics, including number of tumors, tumor size, tumor morphology, vessel density, apoptotic index, etc.

### Diagnostic and Therapeutic Uses

Specific VIPR1-modulating agents are useful in a variety of diagnostic and therapeutic applications where disease or disease prognosis is related to defects in the E2F/RB pathway, such as angiogenic, apoptotic, or cell proliferation disorders. Accordingly, the invention also provides methods for modulating the E2F/RB pathway in a cell, preferably a cell pre-determined to have defective or impaired E2F/RB function (e.g. due to overexpression, underexpression, or misexpression of E2F/RB, or due to gene mutations), comprising the step of administering an agent to the cell that specifically modulates VIPR1 activity. Preferably, the modulating agent produces a detectable phenotypic change in the cell indicating that the E2F/RB function is restored. The phrase “function is restored”, and equivalents, as used herein, means that the desired phenotype is achieved, or is brought closer to normal compared to untreated cells. For example, with restored E2F/RB function, cell proliferation and/or progression through cell cycle may normalize, or be brought closer to normal relative to untreated cells. The invention also provides methods for treating disorders or disease associated with impaired E2F/RB function by administering a therapeutically effective amount of an VIPR1-modulating agent that modulates the E2F/RB pathway. The invention further provides methods for modulating VIPR1 function in a cell, preferably a cell pre-determined to have defective or impaired VIPR1 function, by administering an VIPR1-modulating agent. Additionally, the invention provides a method for treating disorders or disease associated with impaired VIPR1 function by administering a therapeutically effective amount of an VIPR1-modulating agent.

The discovery that VIPR1 is implicated in E2F/RB pathway provides for a variety of methods that can be employed for the diagnostic and prognostic evaluation of diseases and disorders involving defects in the E2F/RB pathway and for the identification of subjects having a predisposition to such diseases and disorders.

Various expression analysis methods can be used to diagnose whether VIPR1 expression occurs in a particular sample, including Northern blotting, slot blotting, ribonuclease protection, quantitative RT-PCR, and microarray analysis. (e.g., Current Protocols in Molecular Biology (1994) Ausubel F M et al., eds., John Wiley & Sons, Inc., chapter 4; Freeman W M et al., Biotechniques (1999) 26:112-125; Kallioniemi O P, Ann Med 2001, 33:142-147; Blohm and Guiseppi-Elie, Curr Opin Biotechnol 2001, 12:41-47). Tissues having a disease or disorder implicating defective E2F/RB signaling that express an VIPR1, are identified as amenable to treatment with an VIPR1 modulating agent. In a preferred application, the E2F/RB defective tissue overexpresses a VIPR1 relative to normal tissue. For example, a Northern blot analysis of mRNA from tumor and normal cell lines, or from tumor and matching normal tissue samples from the same patient, using full or partial VIPR1 cDNA sequences as probes, can determine whether particular tumors express or overexpress VIPR1. Alternatively, the TaqMan® is used for quantitative RT-PCR analysis of VIPR1 expression in cell lines, normal tissues and tumor samples (PE Applied Biosystems).

Various other diagnostic methods may be performed, for example, utilizing reagents such as the VIPR1 oligonucleotides, and antibodies directed against an VIPR1, as described above for: (1) the detection of the presence of VIPR1 gene mutations, or the detection of either over- or under-expression of VIPR1 mRNA relative to the non-disorder state; (2) the detection of either an over- or an under-abundance of VIPR1 gene product relative to the non-disorder state; and (3) the detection of perturbations or abnormalities in the signal transduction pathway mediated by VIPR1.

Kits for detecting expression of VIPR1 in various samples, comprising at least one antibody specific to VIPR1, all reagents and/or devices suitable for the detection of antibodies, the immobilization of antibodies, and the like, and instructions for using such kits in diagnosis or therapy are also provided.

Thus, in a specific embodiment, the invention is drawn to a method for diagnosing a disease or disorder in a patient that is associated with alterations in VIPR1 expression, the method comprising: a) obtaining a biological sample from the patient; b) contacting the sample with a probe for VIPR1 expression; c) comparing results from step (b) with a control; and d) determining whether step (c) indicates a likelihood of the disease or disorder. Preferably, the disease is cancer, most preferably a cancer as shown in TABLE 2. The probe may be either DNA or protein, including an antibody.

### Examples

The following experimental section and examples are offered by way of illustration and not by way of limitation.

### I. E2F/RB Genetic Screen

A genetic screen to identify suppressors genes that when inactivated, decrease signaling through the E2F pathway was designed. In preparation for the screen, the non small cell lung cancer cell line NCI-H1299 was selected for use in the screen, and was engineered to express a construct in which consensus E2F transcription factor binding sites were cloned upstream of a secreted alkaline phosphatase (SEAP) reporter gene. In proof of principle experiments, SEAP secretion was demonstrated to be responsive to serum, and reduced by siRNA mediated inhibition of known positive regulators of the E2F pathway (e.g. CDK4). For the screen, the function of individual genes was inactivated by RNAi using siRNAs designed against each gene and transfected into the NCI-H1299 E2F-SEAP line. The siRNA treated cells were assayed for E2F pathway activity by monitoring changes in the levels of SEAP generated from the E2F reporter or the reduction in the level of a critical phosphorylation event on pRB (p807/811) indicating attenuation of E2F signaling through CDK4 and CDK6.

4 unique individual siRNA duplexes per gene were used to knock down expression of each target. Each siRNA duplex was transfected at a final concentration of 25 nM using OligofectAmine lipid reagent following manufacturers instructions (Invitrogen). A gene was scored as positive if two or more individual siRNAs reduced the amount of E2F driven SEAP secretion or phosphorylated pRb protein in NCI-H1299 E2F-SEAP cells compared to negative control siRNAs. The positive result was repeated in NCI-H1299 E2F-SEAP cells and another derivative of the NCI-H1299 line that contains a SV40 driven SEAP gene to eliminate siRNAs that had a general effect on the transcriptional or secretion machinery. SEAP levels were detected by assaying media removed from the cells at 72 hours post transfection, and the reduction in phospho Rb protein was detected and quantified on the Cellomics Arrayscan fluorescent microscopy platform 72 hours post transfection. The screen resulted in identification of genes that when inactivated decrease signaling through the E2F pathway.

Three cell lines were selected for further validation of the identified targets in addition to the non-small cell lung carcinoma line (NCI-H1299) used in the screen. Two breast adenocarcinoma lines were selected (MDA-MB231-T and MCF-7) as well as one pancreatic adenocarcinoma line (PANC-1). The rationale for selecting these lines was twofold. First, the E2F-Rb pathway is frequently hyper-activated in breast and pancreatic tumors (see Malumbres M and Barbacid M (2001) Nat Rev Cancer. 1: 222-231; Ortega Set al. (2002) Biochim et Biophyis Acta. 1602: 73-87), in which case the targets identified in the screen described above as regulators of the E2F-Rb pathway may have particular relevance. In addition, we determined experimentally that these particular cell lines show a growth dependence on the E2F-Rb pathway; specifically, knock-down of gene expression of known components of the E2F signaling pathway (e.g. CDK4 and CYCLIND1) using RNA interference in these cell lines abrogates cell cycle progression and proliferation.

### II. Analysis of Table I

The E2F/RB modifier VIPR1 identified in the above referenced screens is presented in Table I. The columns “VIPR1 symbol”, and “VIPR1 name aliases” provide a symbol and the known name abbreviations for the modifier of the E2F/RB pathway, where available, from Genbank. The column “VIPR1 Biological Process” provides the cellular processes that the modifier is associated with. The column “VIPR1 Protein Length” provides the length of the amino acid sequence of the modifier. The columns “VIPR1 GI_NA”, and “VIPR1 accno_na” provide the Genbank identifier number (GI#) and the Ref Seq number for the DNA sequences for the VIPR1s as available from National Center for Biology Information (NCBI) and GenBank. The columns “VIPR1 GI_AA”, “VIPR1 accno_aa”, column provide the Genbank identifier number (GI#) and the Ref Seq number for the amino acid sequences for the VIPR1s as available from National Center for Biology Information (NCBI) and GenBank.

TABLE I

| | | | VIPR1 | | | | |
|---|---|---|---|---|---|---|---|
| VIPR1 | | VIPR1 | Protein | VIPR1 | VIPR1 | VIPR1 | VIPR1 |
| symbol | VIPR1 aliases | Biological Process | length: | gi_aa: | gi_na: | accno_aa: | accno_na: |
| VIPR1 | VPAC(1) receptor\|VPAC1 | G-protein signaling, | 457 | 15619006 | 15619005 | NP_004615 | NM_004624 |
| | receptor\|vasoactive intestinal | coupled to cyclic | | | | | |
| | peptide receptor 1\|pituitary | nucleotide second | | | | | |
| | adenylate cyclase activating | messenger; digestion; | | | | | |
| | polypeptide receptor, type | immune response; | | | | | |
| | II\|vasoactive intestinal peptide | muscle contraction; | | | | | |
| | receptor\|PACAP type II | positive regulation of | | | | | |
| | receptor\|VIP receptor, type | cell proliferation; | | | | | |
| | I\|PACAP-R- | signal transduction; | | | | | |
| | 2\|VPAC1\|VIRG\|VIPR\|RDC1\|RCD1\| | synaptic transmission | | | | | |
| | HVR1\|II\|VIPR1 | | | | | | |

### III. Kinase Assay

A purified or partially purified VIPR1 is diluted in a suitable reaction buffer, e.g., 50 mM Hepes, pH 7.5, containing magnesium chloride or manganese chloride (1-20 mM) and a peptide or polypeptide substrate, such as myelin basic protein or casein (1-10 μg/ml). The final concentration of the kinase is 1-20 nM. The enzyme reaction is conducted in microtiter plates to facilitate optimization of reaction conditions by increasing assay throughput. A 96-well microtiter plate is employed using a final volume 30-100 μl. The reaction is initiated by the addition of ³³P-gamma-ATP (0.5 μCi/ml) and incubated for 0.5 to 3 hours at room temperature. Negative controls are provided by the addition of EDTA, which chelates the divalent cation (Mg2⁺ or Mn²⁺) required for enzymatic activity. Following the incubation, the enzyme reaction is quenched using EDTA. Samples of the reaction are transferred to a 96-well glass fiber filter plate (MultiScreen, Millipore). The filters are subsequently washed with phosphate-buffered saline, dilute phosphoric acid (0.5%) or other suitable medium to remove excess radiolabeled ATP. Scintillation cocktail is added to the filter plate and the incorporated radioactivity is quantitated by scintillation counting (Wallac/Perkin Elmer). Activity is defined by the amount of radioactivity detected following subtraction of the negative control reaction value (EDTA quench).

### IV. Expression Analysis

All cell lines used in the following experiments are NCI (National Cancer Institute) lines, and are available from ATCC (American Type Culture Collection, Manassas, Va. 20110-2209). Normal and tumor tissues were obtained from Impath, UC Davis, Clontech, Stratagene, Ardais, Genome Collaborative, and Ambion.

TaqMan® analysis was used to assess expression levels of the disclosed genes in various samples.

RNA was extracted from each tissue sample using Qiagen (Valencia, Calif.) RNeasy kits, following manufacturer's protocols, to a final concentration of 50 ng/μl. Single stranded cDNA was then synthesized by reverse transcribing the RNA samples using random hexamers and 500 ng of total RNA per reaction, following protocol 430-4965 of Applied Biosystems (Foster City, Calif.).

Primers for expression analysis using TaqMan® assay (Applied Biosystems, Foster City, Calif.) were prepared according to the TaqMan® protocols, and the following criteria: a) primer pairs were designed to span introns to eliminate genomic contamination, and b) each primer pair produced only one product. Expression analysis was performed using a 7900HT instrument.

TaqMan® reactions were carried out following manufacturer's protocols, in 25 μl total volume for 96-well plates and 10 μl total volume for 384-well plates, using 300 nM primer and 250 nM probe, and approximately 25 ng of cDNA. The standard curve for result analysis was prepared using a universal pool of human cDNA samples, which is a mixture of cDNAs from a wide variety of tissues so that the chance that a target will be present in appreciable amounts is good. The raw data were normalized using 18S rRNA (universally expressed in all tissues and cells).

For each expression analysis, tumor tissue samples were compared with matched normal tissues from the same patient. A gene was considered overexpressed in a tumor when the level of expression of the gene was 2 fold or higher in the tumor compared with its matched normal sample. In cases where normal tissue was not available, a universal pool of cDNA samples was used instead. In these cases, a gene was considered overexpressed in a tumor sample when the difference of expression levels between a tumor sample and the average of all normal samples from the same tissue type was greater than 2 times the standard deviation of all normal samples (i.e., Tumor−average (all normal samples)>2×STDEV (all normal samples)).

### V. VIPR1 Functional Assays

RNAi experiments were carried out to knock down expression of various VIPR1 sequences in various cell lines using small interfering RNAs (siRNA, Elbashir et al, supra). The following cell lines were used in the experiments: PANC-1 and MDA-MB231T.

Effect of VIPR1 RNAi on cell proliferation and growth. BrdU assays, as described above, were employed to study the effects of decreased VIPR1 expression on cell proliferation using the following cell lines, H1299, MCF7, PANC-1 and MDA-MB231T.

Results: RNAi of VIPR1 decreased cell proliferation in all 4 cell lines.

Standard colony growth assays, as described above, were employed to study the effects of decreased VIPR1 expression on cell growth.

Results: RNAi of VIPR1 decreased proliferation in several of the cell lines tested.

What is claimed is:

1. A method of identifying a candidate E2F/RB pathway modulating agent, said method comprising the steps of:
(a) providing an assay system comprising an VIPR1 polypeptide or nucleic acid;
(b) contacting the assay system with a test agent under conditions whereby, but for the presence of the test agent, the system provides a reference activity; and
(c) detecting a test agent-biased activity of the assay system, wherein a difference between the test agent-biased activity and the reference activity identifies the test agent as a candidate E2F/RB pathway modulating agent.

2. The method of claim 1 wherein the assay system comprises cultured cells that express the VIPR1 polypeptide.

3. The method of claim 2 wherein the cultured cells additionally have defective E2F/RB function.

4. The method of claim 1 wherein the assay system includes a screening assay comprising a VIPR1 polypeptide, and the candidate test agent is a small molecule modulator.

5. The method of claim 4 wherein the assay is a binding assay.

6. The method of claim 1 wherein the assay system is selected from the group consisting of an apoptosis assay system, a cell proliferation assay system, and an angiogenesis assay system.

7. The method of claim 1 wherein the assay system includes a binding assay comprising a VIPR1 polypeptide and the candidate test agent is an antibody.

8. The method of claim 1 wherein the assay system includes an expression assay comprising a VIPR1 nucleic acid and the candidate test agent is a nucleic acid modulator.

9. The method of claim 8 wherein the nucleic acid modulator is an antisense oligomer.

10. The method of claim 8 wherein the nucleic acid modulator is a PMO.

11. The method of claim 1 additionally comprising:
(d) administering the candidate E2F/RB pathway modulating agent identified in (c) to a model system comprising cells defective in E2F/RB function and, detecting a phenotypic change in the model system that indicates that the E2F/RB function is restored.

12. The method of claim 11 wherein the model system is a mouse model with defective E2F/RB function.

13. A method for modulating a E2F/RB pathway of a cell comprising contacting a cell defective in E2F/RB function with a candidate modulator that specifically binds to an VIPR1 polypeptide, whereby E2F/RB function is restored.

14. The method of claim 13 wherein the candidate modulator is administered to a vertebrate animal predetermined to have a disease or disorder resulting from a defect in E2F/RB function.

15. The method of claim 13 wherein the candidate modulator is selected from the group consisting of an antibody and a small molecule.

16. The method of claim 1, comprising the additional steps of:
(d) providing a secondary assay system comprising cultured cells or a non-human animal expressing VIPR1,
(e) contacting the secondary assay system with the test agent of (b) or an agent derived therefrom under conditions whereby, but for the presence of the test agent or agent derived therefrom, the system provides a reference activity; and
(f) detecting an agent-biased activity of the second assay system,
wherein a difference between the agent-biased activity and the reference activity of the second assay system confirms the test agent or agent derived therefrom as a candidate E2F/RB pathway modulating agent, and wherein the second assay detects an agent-biased change in the E2F/RB pathway.

17. The method of claim 16 wherein the secondary assay system comprises cultured cells.

18. The method of claim 16 wherein the secondary assay system comprises a non-human animal.

19. The method of claim 18 wherein the non-human animal mis-expresses an E2F/RB pathway gene.

20. A method of modulating E2F/RB pathway in a mammalian cell comprising contacting the cell with an agent that specifically binds a VIPR1 polypeptide or nucleic acid.

21. The method of claim 20 wherein the agent is administered to a mammalian animal predetermined to have a pathology associated with the E2F/RB pathway.

22. The method of claim 20 wherein the agent is a small molecule modulator, a nucleic acid modulator, or an antibody.

23. A method for diagnosing a disease in a patient comprising:
obtaining a biological sample from the patient;
contacting the sample with a probe for VIPR1 expression;
comparing results from step (b) with a control;
determining whether step (c) indicates a likelihood of disease.

24. The method of claim 23 wherein said disease is cancer.

25. The method according to claim 24, wherein said cancer is a cancer as shown in Table 2 as having >25% expression level.
